(19) United States
(12) Patent Application Publication
(10) Pub. No.: US 2026/0061223 A1
(43) Pub. Date: Mar. 5, 2026

(54) GYROSCOPIC BEAM DELIVERY SYSTEM

(71) Applicant: Zap Surgical Systems, Inc., San Carlos, CA (US)

(72) Inventors: James E. Clayton, San Jose, CA (US); Edward A. Gardner, Ann Arbor, MI (US); Gregory F. Johnson, Rancho Palos Verdes (CA); Cesare Jenkins, Spanish Fork, UT (US); John Adler, San Mateo, CA (US); Robert Wiggers, Belmont, CA (US); Alexander Maslowski, Peachtree City, GA (US); Stephen Wilcox, Los Gatos, CA (US); Chuanyu Zhou, Knoxville, TN (US)

(73) Assignee: Zap Surgical Systems, Inc., San Carlos, CA (US)

(21) Appl. No.: 19/318,131

(22) Filed: Sep. 3, 2025

Related U.S. Application Data
(60) Provisional application No. 63/690,202, filed on Sep. 3, 2024.

(51) Int. Cl. A61N 5/10

(52) U.S. Cl. CPC A61N 5/1082; A61N 5/1031; A61N 5/1071; A61N 2005/1094

(57) ABSTRACT

Methods and systems of radiosurgical treatment include rotating one or both of a first gantry component and a second gantry component while transmitting a therapeutic treatment beam so as to deliver a therapeutic radiation dose to a target tissue of a patient from one or more arcs extending along a treatment sphere. The first gantry component is rotatable about a first axis and a second gantry component is interfaced with the first gantry component such that rotation of the first gantry component rotates both the first and second gantry components about the first axis. The second gantry component is independently rotatable about a second axis. The method includes coordinating movement of the first and second gantry components along the respective first and second axes so as to allow a trajectory of the therapeutic treatment beam to intersect the target tissue from multiple directions along the treatment sphere.

### CROSS-REFERENCES TO RELATED APPLICATIONS

The present application is a Non-Provisional of and claims the benefit of priority to U.S. Provisional Application No. 63/690,202 filed Sep. 3, 2024, the full disclosures of which are incorporated herein by reference in their entirety for all purposes.

### BACKGROUND

Conventional arc therapy generally provides a coplanar system and methods and lack the versatility and range of movement. There is a need for improved systems and methods having more range of movement to avoid critical structures. Two axis systems have been developed, but thus far have provided only therapy at fixed positions. There is further need for such arc therapy systems that are compatible with self-shielded systems and are capable of providing high doses of radiation toward tumors minimizing exposure to the surrounding healthy tissue from harmful amounts of radiation.

### SUMMARY

According to one embodiment, a method of radiosurgical treatment includes rotating one or both of a first gantry component and a second gantry component while transmitting a therapeutic treatment beam so as to deliver a therapeutic radiation dose to a target tissue of a patient from one or more arcs extending along a treatment sphere. The first gantry component is rotatable about a first axis that extends along a patient supported within an interior treatment space and a second gantry component is interfaced with the first gantry component such that rotation of the first gantry component rotates both the first and second gantry components about the first axis. The second gantry component is independently rotatable about a second axis that is transverse to the first axis and intersects the first axis at an isocenter. The method further includes coordinating movement of the first and second gantry components along the respective first and second axes so as to allow a trajectory of the therapeutic treatment beam to intersect the target tissue from multiple directions along the treatment sphere during movement along the one or more arcs.

The method may include various optional embodiments. A path of rotation for at least one of the first gantry component and the second gantry may include comprises a line, an arc, a circle, a great circle, a spherical spiral, a helical spiral, or a rhumb line. The method may further include rotating a collimator wheel mounted within the at least one of the first and second gantry components and in line with the therapeutic treatment beam. The collimator wheel may collimate the therapeutic treatment beam passing through the collimator wheel to the isocenter for treatment of a target positioned within the treatment sphere. The first and second gantry components may rotate independently from the rotation of the collimator wheel. The collimator wheel may rotate along a rotation axis that is perpendicular to an axis of the therapeutic treatment beam. The method may further include adjusting a speed of rotation for each of the first gantry component and the second gantry component. A dose of therapeutic beam delivery to the target tissue may be proportional to the speed of rotation of at least one of the first gantry component and the second gantry component. The collimator wheel may include a plurality of collimator channels including at least a first collimator channel defined within the collimator wheel and a second collimator channel defined within the collimator wheel. The first and second collimator channels may be arranged substantially perpendicular to a rotation axis of the collimator wheel. The method may further include rotating the collimator wheel to align with one or more collimator channels of the plurality of collimator channels with the therapeutic treatment beam, the one or more selected collimator channels corresponding to one or more desired therapy beams along the one more arcs of the treatment sphere. The method may further include rotating the collimator wheel to align with a different one or more collimator channels during treatment where the therapeutic treatment beam is gated during rotation of the collimator wheel between channels. The different one or more collimator channels may be an adjacent channel on the collimator wheel relative to the one or more collimator channels. The method may further include translating a patient table for supporting at least a portion of the patient having the target tissue within the interior treatment space where the patient table is configured to be translated along at least three axes. The target tissue may include one or more distinct regions within a portion of the patient and the method may further include translating the patient table for treating a different region of the target tissue. The method may further include adjusting a hinged head support disposed on the patient table that permits both extension and flexion of the neck of the patient in the sagittal plane, wherein the target tissue is within the head of the patient.

According to another embodiment, a radiosurgical treatment system includes a radiation shield defining an interior treatment space. The radiation shield includes a first gantry component rotatable about a first axis that extends along a patient supported within the interior treatment space and a second gantry component interfaced with the first gantry component such that rotation of the first gantry component rotates both the first and second gantry components about the first axis. The second gantry component is independently rotatable about a second axis that is transverse to the first axis and intersects the first axis at an isocenter. The system further includes a radiation source disposed in at least one of the first and second gantry components and configured to direct a continuous therapeutic beam to a target tissue within the interior treatment space and a control unit operably and communicatively coupled with the radiation source, the first gantry component and the second gantry component. The control unit coordinates movement of the first and second gantry components along the respective first and second axes so as to allow a trajectory of the therapeutic beam emitted from the radiation source to intersect the target tissue from multiple directions along a treatment sphere.

The system may include various optional embodiments. The system may further include a collimator wheel mounted within at least one of the first and second gantry components and in line with the radiation source, the collimator wheel being configured to direct the therapeutic beam passing through the collimator wheel to the isocenter for treatment of a target positioned at the isocenter. The control unit may control movement of the first and second gantry components independently from the rotation of the collimator wheel. The control unit may control a speed of rotation for each of the first gantry component, the second gantry component, and the collimator wheel. A dose of therapeutic beam delivery to the target tissue may be proportional to the speed of rotation of at least one of the first gantry component and the second gantry component. A path of rotation for at least one of the first gantry component and the second gantry component may include a line, an arc, a circle, a great circle, a spherical spiral, a helical spiral, or a rhumb line. The collimator wheel may be rotatable about a rotational axis thereof, the collimator wheel being circular in shape with a diameter and have a plurality of collimator channels including a first collimator channel defined within the collimator wheel and a second collimator channel defined within the collimator wheel. The first and second collimator channels may be arranged substantially perpendicular to the rotational axis of the collimator wheel. The control unit may be configured to rotate the collimator wheel to align with one or more collimator channels of the plurality of collimator channels with the radiation source, the one or more selected collimator channels corresponding to one or more desired therapy beams. The control unit may rotate the collimator wheel to align with a different one or more collimator channels during treatment, wherein the radiation source is gated during rotation of the collimator wheel during treatment. The system may further include a patient table for supporting at least a portion of the patient having the target tissue within the interior treatment space of the radiation shield where the control unit is operably and communicatively coupled with the patient table for moving the patient table along at least three axes. The system may further include a hinged head support disposed on the patient table that permits both extension and flexion of the neck of the patient in the sagittal plane, wherein the target tissue is within the head of the patient.

### DESCRIPTION OF THE DRAWINGS

FIG. 1 illustrates an overview of a treatment system in accordance with some embodiments of the invention.

FIG. 2A shows a side view of the system and interface with a supporting base ring and patient table in accordance with some embodiments.

FIG. 2B shows a front view of the system with an open patient portal in accordance with some embodiments.

FIG. 3 shows a cross section of an example revolving collimator wheel having collimator channels passing therethrough.

FIG. 4 shows an example revolving collimator wheel mounted upon a conical shield, the collimator wheel having magnetic encoder trackers that sense when the wheel has been brought into a desired collimator position.

FIG. 5 shows the apex of an example conical shield, including the exit openings of the collimator channels of the collimator wheel and cameras for monitoring the patient.

FIG. 6 shows an example collimator wheel and associated motor configured to drive between positions, and a linear accelerator that passes radiation through the collimator wheel and associated features.

FIG. 7 shows another example conical shield surrounding a periphery of the collimator wheel to cover orifices of non-aligned channels.

FIG. 8 is a flowchart of an iterative method for path selection, according to embodiments of the present disclosure.

FIG. 9 is an exemplary complicated workspace having an isocenter in the neck, according to embodiments of the present disclosure.

FIG. 10 is an exemplary treatment path, according to embodiments of the present disclosure.

FIG. 11 is a graph of the ratio of angular motion to dose rate, according to embodiments of the present disclosure.

FIG. 12 is a flowchart of a method for treating a patient with a radiosurgical treatment system, according to embodiments of the present disclosure.

FIGS. 13-17 illustrate various exemplary treatment paths, according to embodiments of the present disclosure.

### DETAILED DESCRIPTION

Volumetric modulated arc therapy, or VMAT, is a type of external beam radiation therapy (EBRT) healthcare providers use to treat cancer or the like. With VMAT, the machine rotates around the patient, delivering continuous doses of radiation toward a tumor.

VMAT is also a form of intensity-modulated radiation therapy (IMRT). IMRT delivers a dose over many beam positions where each beam position potentially delivers the dose at a different rate (or strength). This technique allows healthcare providers to direct high doses of radiation toward tumors while mitigating the surrounding healthy tissue to harmful amounts of radiation. For example, VMAT may be used to deliver customized doses of radiation as a machine encircles the patient in one or more rotations, or arcs. VMAT continually adjusts the shape and strength of the radiation beams directed toward the tumor. While such VMAT systems have improved the state of the art regarding precision and speed of delivery, such systems have limitations in regard to the range of available angles. Typically, conventional VMAT systems provide coplanar movement along a fixed plane (e.g., coplanar beam gantries). These limitations may present challenges in treating certain tumor locations, particularly tumors located adjacent critical structures, such as the hippocampus and optic nerve. While there exist other radiation treatment systems having improved range of motion, for example two-axis systems, dynamic therapies, such as VMAT, have not as of yet been implemented in such systems due in part to the complexities in treatment planning and delivery. It would be advantageous to implement dynamic therapies, such as VMAT and arc therapy, into radiation treatment systems having multiple axes of rotation. It would be further advantageous to implement such therapies in self-shielded multi-axis systems, such as those described herein. It is appreciated that these concepts are not limited to the self-shielded systems described herein and may be applicable to any multi-axis system.

The present invention may include a self-shielded radiation treatment system, in particular self-shielded systems having a radiation shield defined by at least two shield components that are balanced and movably interfaced so that the shield assembly rotates about a first axis and one of the shield components rotates about a second axis transverse to the first axis. Such a configuration allows for diagnostic intensity (kV) imaging from multiple directions for tracking the patient's position by rotation of the shielding assembly and further allows for delivery of therapeutic radiation from a range of directions by independent rotation of one shield component along the second axis. Balancing of the shield components about their respective axes of rotation and about a common support and providing shield components of variable thickness substantially reduces the weight of the shielding and makes coordinated and precise controlled movement feasible. This approach also allows for system and drive assembly of more compact size, so as to fit in a standard sized room without requiring an extensive shielding vault common to conventional radiation treatment systems. In some embodiments, this configuration allows the main portion including the radiation shield to be about 3 meters or less in height and width and about 30 tons or less in weight for a 3 MV system. It is understood that the size and amount of shield depends on the radiation energy capacity of the system being used and that the concepts herein may apply to any radiation treatment system. Various embodiments may be further understood by reference to the descriptions and example embodiments described herein.

### I. System Overview

In some embodiments, the radiosurgery system includes the following hardware subsystems and software: mechanical subsystem, patient table subsystem, control subsystem, linear accelerator (LINAC) subsystem, treatment planning subsystem, treatment delivery subsystem, imaging and monitoring subsystem, safety subsystem, and associated software components. It is understood that the integrated control system, safety subsystem and software components may be incorporated into a single control unit or subsystem. Alternatively, these subsystems may include multiple coordinated subsystems or units. Each of these subsystems has corresponding hardware and software components within an integrated robotic system. It is appreciated that embodiments may include some or any combination of the components, or variations thereof.

In the embodiments of the treatment systems described herein, at least two forms of radiation are emitted and detected: (1) mega-volt (MV) X-ray radiation, which is of therapeutic intensity (e.g. sufficient radiation dosage delivery to kill tumor cells) and (2) kilo-volt (kV) X-ray radiation, which is of diagnostic intensity, and is used to track the position of a target (e.g. the patient's skull or body parts) within the apparatus to ensure proper MV beam targeting and delivery. In some embodiments, the MV radiation source is affixed within one movable shield component and the kV radiation source is affixed within the other shield component, the shield components together defining a shielded treatment space around the target and being movable so as to allow imaging of the target with the kV radiation source from a multiple directions and treatment of the target from the MV radiation source from multiple directions.

### A. Multi-Axis Gantry Components

In one embodiment, the radiation shield includes a first gantry component (e.g., first shield component or axial shield) that is movable about a first axial axis, typically horizontal, that is movably interfaced with a second gantry component (e.g., second shield component or oblique shield) that is independently rotatable about a second oblique axis that is transverse to the first axis. In some embodiments, the first axial shield has a generally vertically oriented proximal opening through which the patient table and patient are inserted into the treatment space and a distal angled opening (e.g. 45 degrees) that is movably interfaced with the second shield or oblique shield such that second axis intersects the first axis at a 45 degree angle. In some embodiments, the oblique axis is generally semi-spherical in shape and includes a therapeutic radiation beam emitter and beam stop on opposite sides to allow delivery of a therapeutic radiation beam to the target from a path that encircles the target. Coordinated movement of the shields along the first axial axis and movement of the oblique shield along the second oblique axis allows for a substantially continuous range of the therapeutic beam along a majority of a surface of a treatment sphere with only a small portion of the sphere at the proximal and distal ends being inaccessible.

Typically, in the self-shielded embodiment, the first and second shields are formed of iron or iron alloy, or any suitable shield material. Additional shielding of any suitable material, same or different, may be mounted to the outside of each shield in areas exposed to higher radiation levels. In some embodiments, the treatment radiation source or treatment radiation beam emitter (e.g., LINAC) is affixed within one of the shields, while the diagnostic imaging radiation source is disposed within the other shield. The shield components may further include counterweight mounted so as to balance each shield component or assembly about their respective axes of rotation.

### B. Mechanical Subsystem

In another embodiment, the self-shielded radiation treatment system includes a mechanical subsystem that coordinates movement of the movable shield components to facilitate imaging and treatment from multiple directions. In some embodiments, the mechanical subsystem includes a two degrees of freedom rotary electro-mechanical shield assembly that houses the LINAC and imaging subsystems. Its purpose is to move the LINAC so as to direct the high-energy treatment beam generated by the LINAC to point at the system isocenter (e.g., where the target or tumor will be located) in a precise fashion. The two degrees of freedom allows a variety of angles of approach for the treatment beam to be achieved.

In some embodiments, the system includes a rotary shell attached to the mechanical rotatable shield assembly that houses a patient table subsystem with a vertical door at the end of the rotary shell that moves up and down. These two mechanisms serve as the patient entry/exit to the system.

In various embodiments described herein, the mechanical subsystem includes:
1. Two moving radiation shields (e.g., axial shield & oblique shield) which support the radiation treatment beam generator components.
2. A central main base ring that supports the axial and oblique shields, typically through a bracket with the two-rotation axis bearing.
3. A patient entry assembly that supports patient table and additional static radiation shield.
4. Rotary shell along with a vertical radiation shield that compose the entrance and exit to the treatment space.

In order to provide a head and neck treatment beam solid angle that would exceed 2 π steradians, the treatment radiation beam generator components are integrated on the oblique radiation shield supported within an oblique support bracket with two degrees of freedom. In some embodiments, the system is configured to provide the largest solid angle coverage. Those degrees of freedom are transverse to each other. In the embodiments described, those degrees of freedom are pure rotations along:
1. An axial axis which is horizontal (i.e., perpendicular to gravity)
2. An oblique axis that is oriented 45 degree with respect to the axial axis. This 45-degree angle remains constant between those two axes.

In one embodiment, those two axes of motion of the two movable shield components intersect at the isocenter of the treatment system in which the patient tumor is located. In some embodiments, a moving patient table subsystem may be used to ensure the target remains at the isocenter. In the described embodiments, the therapeutic radiation beam generator (i.e., LINAC) is integrated such that the beam aims at the isocenter for any position of the radiation shields. In some embodiments, the two movable shield components are supported by a common support, typically a main base ring. This may be accomplished by balancing the two movable shield components about the support, as described further below. Typically, the base ring is of cast construction and may be anchored to the ground using structural anchoring methods. This configuration reduces the footprint of the overall treatment system. The axial rotation of the radiation shields may be accomplished using a large slew ring ball bearing. The outer ring of the slew ring bearing may be mounted to the main base ring. The axial bearing is sized to provide the required stiffness to minimize the deflection of the bearing due to the applied external forces (i.e., gravity and magnetic attraction load induced by the linear motor). The inner ring of the axial bearing is clamped between two rotating assemblies: the axial radiation shield and the oblique radiation shield-mounting bracket (“treatment bracket”). The axial shield along with the treatment bracket revolve together around the axial bearing axis driven by a linear ring motor that includes a set of linear magnet crescents mounted at the periphery of oblique shield mounting bracket, a large cage-like structure that encloses the oblique shield and associated electronic equipment, and which is visible from the posterior side of the machine. To provide enough torque to rotate the axial assembly, the system uses two motor coil assemblies that include a set of six coils each. Those two assemblies are located side by side at the bottom of the system main ring assembly to provide a counter moment to the moment due to the moving assembly gravity load. The axial motion position, velocity and acceleration is controlled using one ring scale along with two redundant encoder head sensors. While a particular configuration of the axial bearing and motor assemblies are described here, it is appreciated that various other configurations and any suitable motor assemblies may be used to provide rotation of the axial shield and oblique shields along the axial bearing axis in keeping with the described concepts.

In another embodiment, the oblique shield is independently rotatable along a second axis transverse to the axial bearing axis. In the embodiments described herein, the oblique shield is mounted on the oblique treatment bracket through the oblique slewing ball bearing, which has a rotation axis oriented at 45 degrees with respect to the axial bearing axis. The oblique slew ring ball bearing is sized to provide a required stiffness to minimize the deflection of the bearing due to the external forces that include gravity and the magnetic attraction load induced by the linear motor. In some embodiments, the treatment bracket is one part on which both axis bearing lodgings are machined, which avoids stack up of tolerances if using more than one bracket and ensures optimal accuracy. The radiation treatment beam generator components are integrated on the oblique radiation shield such that the treatment beam generator may be rotated entirely around the target. To provide enough torque to rotate the oblique shield assembly around the oblique axis, the rotating shield utilizes two identical motor coil assemblies. Typically, each motor coil assembly includes a set of six motor coils. In this embodiment, oblique shield assembly and the motor coil assemblies are mounted symmetrically to vanish the moment due to the coil/magnet attraction force. This attraction force induces a constant compressive axial load for the bearing, which is beneficial. In some embodiments, the oblique motion position, velocity, and acceleration may be controlled using one ring scale along with two redundant encoder head sensors. It is appreciated that various other configurations may be used.

In yet another embodiment, the treatment system may include a patient entry assembly to facilitate entry of the patient into the shielded treatment space defined by the axial and oblique shields. Typically, the patient entry assembly remains static with respect to the rotating axial and oblique radiation shields. In the embodiments described herein, the patient entry assembly is mounted to the main base ring and includes a radiation shield block as shown in the accompanying figures, a mount for the patient table, and a rotary shell that serves as a radiation shield rolling door for the self-shielded capsule system. The rotary shell rotates around the patient table mount by means of a geared slewing ball bearing mounted to the patient entry bracket. The geared bearing may be rotated using a pinion driven by a geared electrical motor assembly. The geared motor is equipped with a brake that is activated in the loss/absence of electrical power to the system to maintain the rotary shell in its position. A radiation-shielded vertical door assembly moves vertically to open/close the patient entry of the rotary shell. This radiation shield vertical door moves up and down using an actuator. In some embodiments, the door system is configured such that in the case of a power loss, the door may be moved down, and the patient entry opened without power to allow the patient to be removed. For example, the door may be opened without power in a controlled manner by manually opening a pressure relief valve, releasing the energy induced by the large vertical gravity load.

In still another embodiment, a collimator assembly may be mounted to the LINAC subsystem. An example of collimator assemblies suitable for incorporation into the treatment systems are detailed further in PCT Application No. US2017/038256. In such embodiments, the collimator assembly is spherical/cylindrical and is centered on the LINAC radiation beam axis. This mechanical axis intersects at the isocenter. Typically, the collimator assembly provides a selectable set of different collimator sizes. To achieve this later, those different collimator sizes may be designed into a revolver. To select the different collimator size, the revolver rotates via a harmonic drive (e.g., geared) electrical motor around an axis perpendicular to the LINAC radiation beam axis. This geared motor may be integrated onto the main housing, for example, as shown in the accompanying figures. A set of two redundant rotation encoder head sensors along with a scale may be mounted to the revolver to provide the position control feedback of the revolver to align each collimator size with the LINAC beam axis. In some embodiments, an additional “collimator size” sensor may be used to determines the proper position/alignment of each collimator size. The collimator assembly may be mounted onto the oblique shield, aligned with the beam axis intersecting the isocenter. Across from this collimator assembly, a radiation beam stop may be mounted. More shielding may be integrated around the LINAC to shield the backward scattering radiation from the LINAC target. The collimator and the radiation beam stop may be constructed of any suitable material, although typically, they are formed of tungsten or a tungsten alloy, which allow for a collimator and beam stop of reduced size for incorporation into the described treatment systems.

### C. Patient Table Subsystem

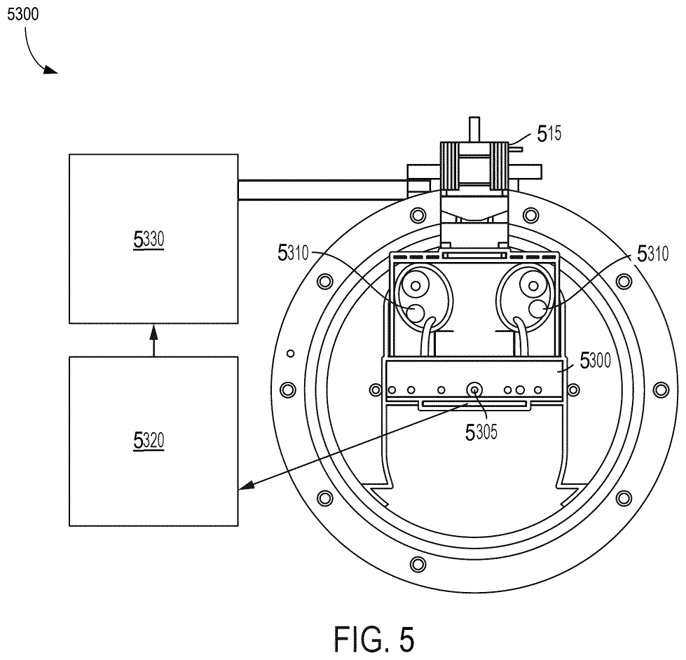

In some embodiments, the treatment system includes a movable patient support table that is sufficiently movable along multiple axes to allow at least the portion of the patient having the target to be positioned within the treatment space defined by the movable shield components. In the embodiments described herein, the patient table includes a three-axis mechanism that serves at least two objectives—first, to provide a bed support on which the patient may lie down comfortably during treatment, and second, to accurately maintain position, in three dimensions, of a desired point in the head and neck region at the isocenter, where radiation will be delivered. To accomplish these objectives, the patient table may be defined by multiple components that allow for movement of the patient along multiple axes. In some embodiments, the patient table has at least four sub-sections—lower cart, upper cart, pitch plate and patient bed. In some embodiments, the table may be that shown and described in FIG. 5 of U.S. Pat. No. 10,499,861 or a modified version thereof. The lower cart has the function of moving the patient between treatment and extraction positions. To set up the patient for treatment, patient table extends in a linear rolling fashion, from the shield assembly to the outside of the patient portal. The upper cart, pitch plate and patient bed together provide the motion needed to accurately position a point in the head and neck at the isocenter. The upper cart houses the control components for the patient table. The lower cart, upper cart and patient bed are actuated by linear motors, while the pitch plate is actuated by a rotary motor with a lead screw arrangement. A head support portion of the patient table may include facets with which to secure commercially available radiation face masks, and the patient bed may further include a restraining strap to prevent patient body from significant movement during delivery. In some embodiments, the head portion may further be configured to pitch the patient's head to further increase the available treatment range of the LINAC. In some embodiments, the table is modified with a head rest that may give a patient's head extension or flexion to increase the amount of solid angle available for treatment of certain tumor sites. It is appreciated that the treatment system may utilize a multi-axis patient table without the movable head portion as well.

In some embodiments, the patient table has at least five sub-sections - table base, lower cart, upper cart, pitch plate and patient bed. The table base provides a fixed support for the patient table. The table base acts as the interface between the patient table and the mechanical subsystem, that is, the patient table may be attached to the mechanical system using the table base. The table base may also provide additional shielding. The lower cart performs linear motion (Y1 axis) and serves the function of moving the patient between treatment and extraction positions. The lower cart may be driven by any suitable motor. In some embodiments, the lower cart is driven by a linear motor using direct drive with no transmission. Such a configuration avoids any power loss that happens in case of a mechanical transmission like a gear train. Direct drive is also more responsive. In addition, this configuration is back drivable which may be an important safety feature. In case of power failure, the lower cart may be pulled out manually due to its back-drivability. The upper cart performs linear motion (Y2 axis), which along with the pitch plate and patient bed, provides the motion needed to accurately position a point in the head and neck region at the isocenter. The upper cart may be driven by any suitable motor, for example, a linear motor having the advantages as discussed above. The pitch plate may tilt up and down (pitch axis) and, along with the upper cart and patient bed, provide the motion needed to accurately position a point in the head and neck region at the isocenter. In some embodiments, the pitch plate is tilted up and down using a lead screw driven by a rotary motor. Such a configuration is advantageous since the lead screw is not back drivable and hence will hold its position during an accidental power loss. The patient bed may perform an arced side to side rotation (yaw axis) motion driven by any suitable motor. In some embodiments, the yaw rotation is directly driven by a curved linear motor, which, along with the upper cart and pitch plate, provides the motion needed to accurately position a point in the head and neck region at the isocenter. Such a configuration is back-drivable as well. From a targeting perspective, the described patient table configurations serve to locate a spatial point {x, y, z} within the patient, at the isocenter (three degrees of freedom provided by the three axes).

In some embodiments, the patient table includes one primary and one redundant encoder per axis. The link between system computer and motion controller may be directly wired or Ethernet. The link between motion controller and motor drives may be directly wired or EtherCAT. Motor drives, motors, and encoders are typically located in patient table assembly.

In another embodiment, the treatment planning subsystem is configured to be compatible with the patient table subsystem at the reference frame level in the world coordinate system. The treatment delivery subsystem accounts for any possible changes to the patient setup before treatment is delivered by moving the patient with the patient table so that the anatomic target area is at the physical isocenter of the system, and this function is continued throughout treatment to compensate for any patient movement detected by the imaging subsystem.

### D. Integrated Control Subsystem

In yet another embodiment, the system may include a motion control subsystem and a LINAC control subsystem that collectively works to coordinate device position sensor input and therapeutic radiation delivery monitoring input with the various motors on the mechanical subsystem and patient table subsystem to move the various components of the mechanical subsystem and patient table to ensure that each radiation beam from all portions of the delivery arc is properly aimed at the target within the patient's body, and at assigned trajectories to the same at the proper times to deliver the prescribed treatment. This ensures that radiation goes only to the anatomical target. This also enables shielded patient port to be closed when necessary and open when necessary. These objectives may be achieved by interactively networking the mechanical subsystem, LINAC subsystem, treatment planning subsystem, treatment delivery subsystem, imaging and monitoring subsystem, patient table subsystem, and control and safety subsystem.

Communications between the computer, sensors and actuators may be implemented by any suitable means, for example using an EtherCAT real-time network (e.g., Beckhoff, Lenze, Sanyo-Denki, ACS) to monitor peripheral sensors and devices and control systems and actuators from a main computer system.

### E. Linac Subsystem

In still another embodiment, the system includes a LINAC subsystem that produces the treatment beam. Typically, the LINAC subsystem is affixed within the second movable shield component (e.g., oblique shield component) such that movement of the shield component rotates the LINAC entirely about the target. The system may be configured to generate a charged particle treatment beam or a photon treatment beam. In some embodiments, the system uses a LINAC to produce a treatment beam with a nominal energy of 1 to 5 MeV, preferably 2 to 4 MeV, more preferably 3 MeV for a dose rate within a range of 1400 to 1600 cGy/min. In various embodiments, the dose rate of the delivery LINAC is at least 2100 cGy/min. For example, in at least some embodiments, the dose rate is greater than or equal to 2100 cGy/min. In some embodiments, the systems is configured to produce a treatment beam with an energy within a range of 2-3 MV for a dose rate within a range from 1000 to 2000 cGy/min. In one embodiment, the treatment beam is collimated to produce one a suitable treatment beam. In some embodiments, the LINAC subsystem is configured to produce a range of differing field sizes, for example field sizes within a range of 3 mm is 4 to 50 mm. In the embodiment shown, the LINAC subsystem includes eight available field sizes, such as diameters of 4 mm, 5 mm, 7.5 mm, 10 mm, 12.5 mm, 15 mm, 20 mm, and 25 mm at the Source to Axis distance (SAD) of, for example 450 mm. In further embodiments, the LINAC subsystem includes any number of holes such as nine holes, ten holes, eleven holes, etc. In one embodiments, the LINAC subsystem includes nine available field sizes such as diameters of 3 mm, 4 mm, 5 mm, 7.5 mm, 10 mm, 12.5 mm, 15 mm, 20 mm, and 25 mm. It is appreciated that the LINAC subsystem may be configured to provide a collimated beam at various other diameters as needed for a particular therapy or target size. Each of the field sizes may be circular and symmetric, or may be square, rectangular, or any other shape desired. In some embodiments, the LINAC comes with a variety of safety interlocks which are integrated into the control and safety subsystem.

The LINAC subsystem may include any suitable components needed to deliver a given radiation therapy to the target from multiple directions. In the embodiment described herein, the LINAC subsystem includes the LINAC, motorized secondary collimators, magnetron, solid state modulator, gun drive power supply, RF waveguide, dosimeter board, automatic frequency control (AFC) board, and LINAC control board. In some embodiments, the LINAC is configured such that the single photon beam energy is in the range of 3 MV. In some such embodiments, the depth dose =40±2% for 2.5 cm circular field size at 45 cm Source to Surface Distance (SSD) with an ionization ratio of d₂₀₀/d₁₀₀=0.5. In other such embodiments, the depth dose maximum (Dₘₐₓ) is 7+/−1 mm. In some embodiments, the dose rate is 1500+/−10% MU/min at 450 mm Source to Axis Distance (SAD); 1MU=1cGy at SAD=450 mm, 25 mm field size at Dₘₐₓ. In various embodiments, the dose rate of the delivery LINAC is at least 2100 cGy/min as described above. In some embodiments, the LINAC subsystem includes a custom LINAC control board, automatic frequency control board (AFC), dosimeter and dosimeter board for incorporation into a self-shielded treatment system in accordance with embodiments of the present invention.

### F. Imaging & Monitoring Subsystems

In order to meet certain radiosurgical precision requirements, the treatment system may include an imaging and monitoring subsystem that provides a means of tracking the position of the tumor with respect to the system isocenter. In some embodiments, for tracking purposes, the self-shielded capsule is equipped with a kV tube with an X-ray supply, along with a kV imaging detector. Prior to activating the radiation beam, the imaging and monitoring subsystem takes images of the patient's head and verifies that the tumor is in position (e.g., located at the isocenter). In case of a position discrepancy, the patient table automatically moves to compensate for the position discrepancy bringing the anatomical target into the system isocenter.

In some embodiments, to monitor patient position (and inferentially with respect to the LINAC, the imaging and monitoring subsystem includes an imaging radiation source fixed in a first shield component and a radiation detector affixed in a second shield component opposite the radiation source. In the embodiment described herein, the imaging radiation source is a kilo-voltage X-ray source, and the imaging radiation detector is an amorphous silicon flat panel detector, although it is appreciated that any suitable imaging radiation source and detector may be used in other embodiments. During treatment, the subsystem obtains images of the patient anatomy episodically, determines patient movement, if any, and directs the patient table subsystem to adjust the patient position to position the tumor at the system isocenter as needed.

In one embodiment, the imaging and monitoring subsystem is configured with a sequential view tracking methodology. The system performs imaging, in which at least two images are obtained sequentially from the imaging and monitoring subsystem to determine a position of the target. In the embodiments described herein, the kV tube and detector are used to acquire live patient image. In contrast, conventional systems, such as CyberKnife system or Brainlab system, use two sets of imaging devices for stereo image tracking. Stereo image tracking combines tracking results from each imaging system to form six degrees of freedom results. To have a quick and accurate solution for patient alignment and tracking, the embodiments described herein utilize a moveable imaging subsystem to obtain sequential views from different perspectives toward the patient's head. For example, the imaging radiation source may be used while the first shield component is at a first position to obtain a first image, and then the first shield component may be rotated to move the imaging device to a second position to obtain a second image from another perspective, the first and second images being used to determine alignment of the target with the isocenter. The system may be used in both initial patient alignment and tracking during delivery. While embodiments described herein include a single diagnostic radiation source, it is appreciated that in some embodiments, multiple sources at different locations on the first shield components may be used.

Patient tracking is done with single images, where kV images are taken at some consistent frequency, say 45 seconds as a maximum. At this frequency, single images are used to calculate patient position in 5 DOF. Based on the position, the tumor will be automatically moved if the offset is below a given threshold, above that limit a second image is taken to resolve all 6 DOF. In another implementation, images acquired previously are used to help resolve to 6 DOF and thus improve positional accuracy.

An exemplary imaging method utilizing sequential view tracking methodology may include the following steps:
1) Obtain first image at a first location of the diagnostic imaging system and correlate the X-ray image with digitally reconstructed radiograph (DRR) image to get accurate 2D translation (TX1 and TY1).
2) Move the diagnostic imaging system (typically by movement of a gantry or shield assembly on which the system is mounted) to at least a second location (may be a single axis motion, or combination of two axes), and correlate again to get second accurate 2D translation (TX2 and TY2).
3) Based on displacement matrix (i.e., rotation) between those two locations, combine two results (TX1/TY1, TX2/TY2) to calculate depth result of (TZ2).
4) Combine depth results (TZ2) with current location result (TX2/TY2) to form a 3D translation result (TX2/TY2/TZ2).
5) If any table motion happened between those two imaging locations, the displacement matrix (in Step 3) may also include table translation.
6) To get an accurate and robust TZ2 result, several approaches may be used:
   a. A minimal rotation angle reduces noise. (e.g., 20 degrees or greater, typically an angle between 40 and 70 degrees, preferably about 60 degrees).
   b. A heuristics approach may be used to combine more than two images. For example, the sequential view results may be generated from the last image with all historical (previous, previous—1, previous—2, . . . ) images. Only the last few are used to avoid potential patient motion, and any outliers are removed to find the average results.
   c. An iterative approach may also be used between (3) and (4).
7) In addition to 3D translation result, 3D rotations may be calculated separately after initial position is close to the aligned position using coordinate descent or related optimization approach.
   a. Search RotationX to find best match within a range (for example, −5 to +5), while fixing RotationY and RotationZ.
   b. Keep optimized RotationX, then search for optimized RotationY and RotationZ until new best values are found.
8) After initial rotations are found, optimization may be done, and steps 1 to 4 are optionally repeated for patient alignment.

In a second exemplary method, the system finds the position and orientation of a computed tomography (CT) that matches the one or more kV images to DRRs by minimizing a cost function using a multivariate solver. Once the current CT position and orientation has been determined, the table move that will bring the isocenter to the machine center may be calculated and applied. Once this match is found, the patient may be moved to position the isocenter in the machine center.

The judgment of how well the DRR matches the kV image may be based on a comparison of intensity or edge-detected images or images that have been processed in other ways to reduce noise or improve the matching process.

A multivariate minimization algorithm like the Nelder-Mead gradient descent solver may be used to find the DRR parameters describing patient translations and rotations that result in the best match with the acquired kV image. The Nelder-Mead method samples the cost function by creating DRRs with different CT locations and orientations. An algorithm determines the location of the next sample based on the values of the current cost function samples. By this method, the minimum of the cost function is determined without creating DRRs for the entire six-dimensional space. Depending on the information available, this solver may be used to find solutions in six-dimensions (3 translations and 3 rotations), just 3 translation or 2 translations (particularly when a single kV image is available).

### G. MV Radiation Beam Monitor Subsystem

In another embodiment, the system monitors the MV (therapeutic) radiation beam during treatment with a MV radiation beam monitor subsystem. The purpose is not to determine the position of the patient (as with the kV beam), but rather to verify and quantify the radiation intensity that passes through the patient. When captured after having passed through a patient (and knowing how much radiation was output by the LINAC), the residual radiation may be correlated with how much radiation the patient absorbed by comparing to the amount of radiation expected to be passed through the patient.

In some embodiments, the output of the MV radiation beam is measured by use of a MV radiation beam monitor subsystem that includes a scintillating membrane and one or more cameras that detect light from the scintillating membrane and output a corresponding signal. The resultant digitized signal is then processed through video signal processing electronics and fed into a system computing unit. The system computing unit may then determine the dose data, beam profile data and the beam positioning data. One potential advantage of this embodiment is higher spatial resolution of the data since a high-resolution camera may be employed. A second advantage of this embodiment is simplicity and cost.

In some embodiments, the MV radiation beam monitor subsystem includes a removable MV radiation beam monitor unit, which is to be replaced before each treatment to ensure the MV radiation beam monitor performs properly and does not degrade with re-use. In some embodiments, the unit includes a removable MV detector camera with scintillating sheet. Typically, the scintillating sheet is made of phosphor Gd₂O₂S:Tb (GOS) on a silicone (PDMS) matrix and cast into a sheet. For every beam delivered, corresponding images are stored on the computer. The camera may be used a single time and replaced because the CCD camera degrades with MV radiation; a new factory-calibrated camera ensures accurate reading each time. The removable MV radiation beam monitor unit includes one or more coupling and/or alignment features to ensure consistently accurate spatial placement. The alignment features may include positive stops and positive locking mechanisms, for example, including magnets, latches, pegs, mortices, or any suitable means. In some embodiments, the shield component in which the removable MV radiation beam unit is attached includes a contoured region that facilitates a desired placement of the unit or orientation, for example, the scintillating sheet being substantially perpendicular to the MV radiation beam. The contoured region is dimensioned to receive the removable unit and may include the positive stops and positive locking mechanisms therein to facilitate secure attachment of the removable MV radiation beam unit at a desired position, alignment, and orientation. In addition to CCD, the imaging device include CMOS cameras, or any other digital imaging device.

A substantial deviation from expected dose to the measured dose will indicate an anomaly and the system will be shut down via the integrated control subsystem. This subsystem may include various inter-connect cables and other ancillary devices. Some of the ancillary devices include cameras and an intercom for the user to monitor and interact with the patient during the treatment. The system provides real time monitoring of the dose prescribed to the patient for each LINAC position. This monitoring feedback feature ensures that the treatment planning is delivered as prescribed and, in one embodiment is implemented with a megavoltage (MV) imager that undergoes a change in response to absorbed radiation, and may be replaced with a new, factory-calibrated unit.

In some embodiments, the MV radiation beam monitor subsystem includes a removable single-use MV detector with silicon diode-based MV detector. In some embodiments, the single-use MV detector includes a scintillator and one or more photodiodes. Such a detector allows use of various techniques for in-situ radiation intensity measurement to quantify the quality of the treatment and provides data on in-situ positioning of the radiation beam, intensity distribution within the beam without the patient in the beam path, and residual beam with the patient in the beam path. The dose delivered for each site may be determined using this measurement for verification, with or without the patient in the beam path. The measured residual dose may be determined from a therapeutic beam detector unit and measured at multiple points. The theoretical value of the residual dose that is determined by the treatment planning system may be compared with a measured residual dose value using this technique for validation of the treatment and/or to assess treatment delivery quality. Validation or quality determinations may be recorded and used to adjust subsequent therapy delivery. In some embodiments, the output of the diode array is amplified, digitized, and fed to a smart controller where the data is sorted and scaled and sent to an onboard system computer. The system computer with additional processing may be used to determine dose delivered data, the beam profile data, and beam positioning data. The LINAC that provides the high energy X-Rays, is modulated with a very small duty cycle (e.g., 500:1 duty cycle). Typically, the beam is only on for less than 1/300^{th} of the time (e.g., 1/500^{th} of the time), the rest of the time the beam is off; however, the time constant of the scintillator may be almost 3 orders of magnitude slower. The signal acquisition is synchronized with the on time of the radiation pulse to maximize the amount of signal to be read. This technique helps with the signal to noise ratio for low-level signals. To characterize the therapeutic radiation beam quality, spatial position and intensity measurements of the beam may be carried out. These values may be used to characterize the beam quality during the QA period of the system; and may be used during the treatment to provide beam position and intensity distribution data and may be used with a secondary dose measurement to validate actual dose delivered is desired and to quantify the quality of the treatment. For example, a residual dose measurement of the beam after passing through the patient may be used to compare with a calculated residual dose to validate the quality of the treatment. By analyzing the residual beam intensity distribution along with the CT data, one may be able to determine the positional accuracy of the beam with respect to the tumor during the treatment.

In one embodiment, the MV radiation beam monitor subsystem includes a scintillator positioned incident to the high energy X-ray radiation such that the radiation excites the scintillator atoms that in turn produce emission of photons in the visible range. The visible light intensity is proportional to the radiation intensity. In some embodiments, a series of photodiodes are used to convert the visible light to electrical signals as an input to the system computer. The scintillator convers the radiation to visible light in the range of the photodiode's detection range. The photodiode array may be placed immediately after the scintillator to maximize the signal level and improve the signal to noise ratio. A number of diodes sufficient to cover the beam diameter should be used to provide a beam intensity profile measurement. There are various photodiode array configurations that may be used. In some embodiments, the photodiode array utilizes 16 element diodes per chip and a sufficient number of chips to cover the entire beam using the largest collimator aperture. The electrical signal from the diode arrays is then amplified and digitized and fed to a computer. The computer software digitally processes the signal and produces dose measurement for validation during the treatment, as well as producing the beam intensity profile, and the XY position data. The XY positional data may be used to validate the accuracy of the beam's position on the tumor and to report possible errors.

Other light detection methods such as CMOS or CCD camera may also be used. In some embodiments utilizing CCD cameras, due to the small size of the camera's active area, such configurations typically use multiple optical components to project the image to the camera. The optics typically require that the CCD cameras be placed some distance away from the scintillator to allow room for focusing. The signal intensity received by the CCD camera is calibrated to a known radiation intensity, thereby compensating for any loss of light occurring in the interposed distance between scintillator and CCD.

### II. Detailed Examples

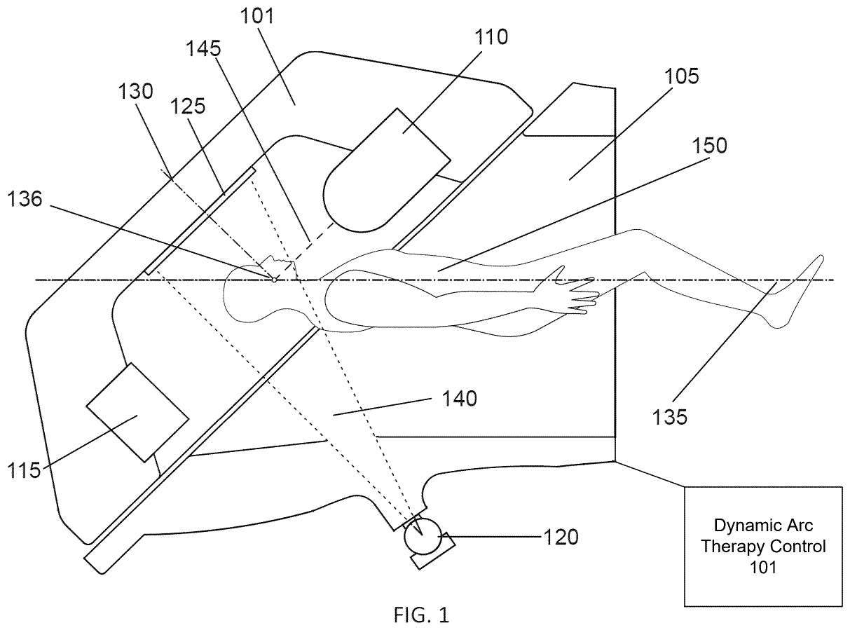

FIG. 1 illustrates an overview of an exemplary multi-axis gantry system 100 designed as a self-shielded treatment system, which includes the mechanical subsystem, the largest piece of hardware in the system and a dynamic therapy control unit 101. While this embodiment is designed as self-shielded system, it is appreciated that multi-axis gantries may encompass non-shielded systems as well. This system includes a shield that includes two movable shield components, oblique shield 110 and axial shield 115 oblique shield 110 rotating on oblique axis 130 and axial shield 115 rotating on axial axis 135. Upon rotation of axial shield 115, both axial shield 115 and oblique shield 110 are rotated about axis 135, while oblique shield 110 is independently rotatable about an oblique axis 130 that is transverse to the axial axis 135. Patient 150 is lying within the apparatus on a patient table (not shown) substantially aligned along axial axis 135 with target at isocenter 136 located at intersection of axial axis 135 and oblique axis 130. Mounted on the inner surface of oblique shield 110 is LINAC 110 that produces MV radiation therapy beam 145 that passes through patient 150 and received by MV radiation detector 115 also mounted upon the inner surface of oblique shield 110. Mounted upon the internal surface of axial shield 115 is KV radiation emitter 120, which is used for real-time X-ray image-based position sensing by passing its beam 140 through patient 150 to kV radiation detector 125 mounted on the internal surface of oblique shield 110. It is noted that the shield components depicted are of solid construction and hatch lines have been omitted merely for clarity.

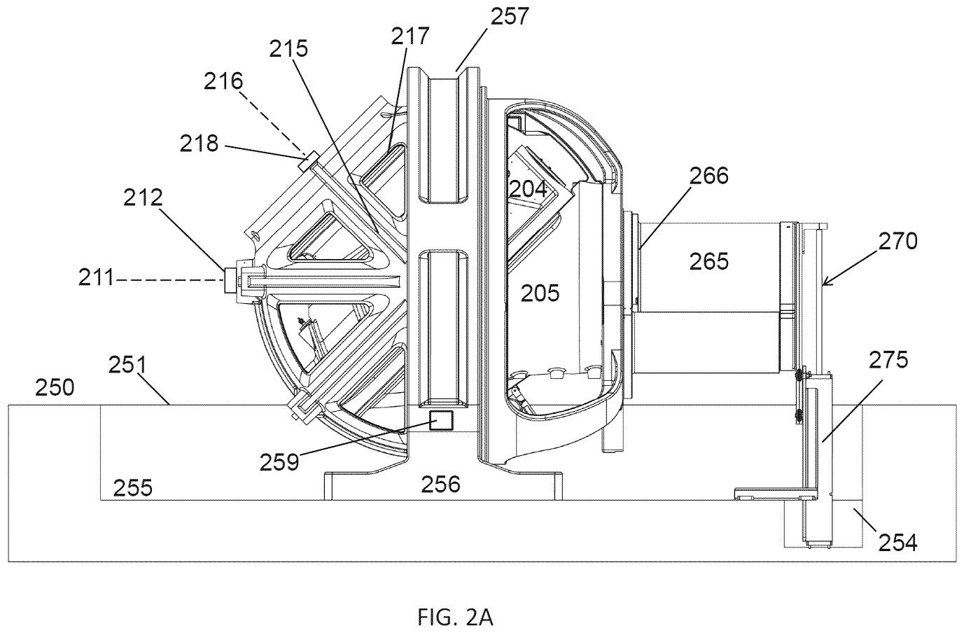

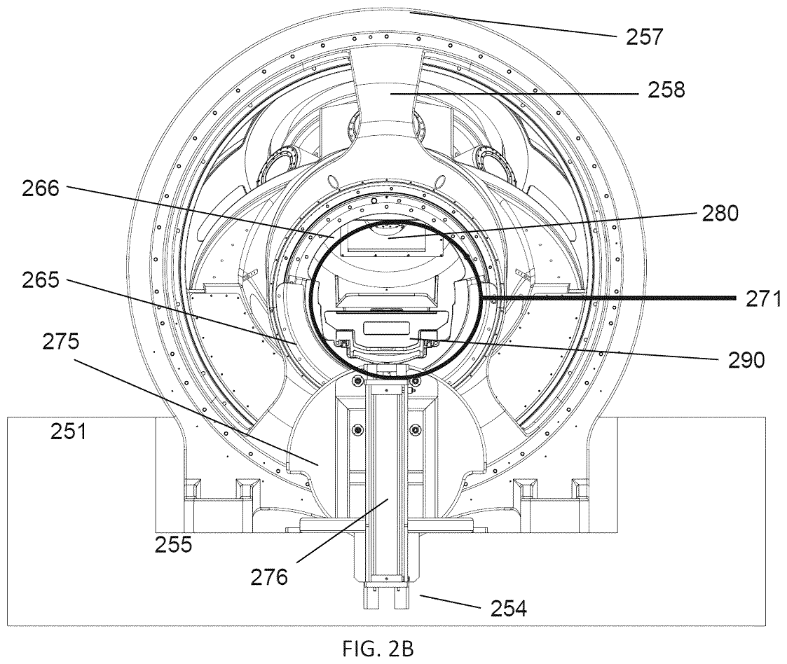

FIG. 2A shows the mechanical subsystem of the treatment system in greater detail as viewed from the side. In this embodiment, the subsystem is housed in a floor pit, although it is appreciated that the subsystem may also rest on a floor surface without a pit assuming sufficient overhead clearance. The axial axis 211 turns via the axial bearing assembly (not visible as it is obscured by base ring) with multichannel electrical and electronic supplies rotationally commuted by axial slip rings 212. Axial shield 205 turns on axial axis 211 around the torso of the patient within. Oblique axis 216 turns on oblique bearing assembly 217 with multichannel electrical and electronic supplies rotationally commuted by oblique slip rings 218. Oblique shield 204 is covered by and enclosed with system electronics by oblique support bracket 215. Shell 265, which rotates on shell bearing 266, covers and shields the entry portal from above the patient table base 260 when the system is in the closed, shielded configuration. Portal is the entryway into the interior of the device that encloses the upper 2/3 of the patient table when door 275 and shell 265 are in the closed and shielded configuration.

In this embodiment, the treatment system sits within a formed concrete pit base 255 approximately two feet deep. The pit serves as additional radiation shielding for the lower portion of the device and advantageously places the patient bed (not shown here) at a comfortable height for seating and bringing patients in and out of the device to the floor level. The pit also reduces the ceiling height required for the apparatus in the room and makes the apparatus more aesthetically appealing by appearing smaller. Portions of the pit not occupied by the apparatus itself may be covered by flooring 251 that meets the normal floor level 250 of the apparatus. The entire mechanical apparatus is held together chiefly by a strong central base ring 257 which is anchored to the concrete at the bottom of pit base 255 with ring base 256 and balances the weight of oblique shield 204 and axial shield 205 and other massive components of the system. It is appreciated that ring base 256 may be an integral portion of base ring 257, as shown here, or may a separate component attached thereto. A deeper extension of the pit 254 accommodates the vertical travel needs of door 270 at its fully opened position.

In this embodiment, the treatment system includes proximity detectors 259 disposed near the base on each side to detect proximity of a person so as to effect an automatic shut-off of radiation and motion upon unauthorized entry of a person into an immediately surrounding zone so as to prevent unintended exposures to radiation or contact to moving system parts during treatment. In some embodiments, the proximity detector has a detection range of at least 180 degrees, typically up to 270 degrees such that one proximity detector on each side of the treatment system effectively covers a zone extending around the entire system. Alternatively, a single proximity detected with a 360-degree range may be positioned above the entire system. The area covered by the proximity detectors may be marked by a boundary. Such a configuration may allow the area outside the boundary to be an uncontrolled area since there is negligible risk of unintended exposure since the system will shut off if the boundary is crossed.

FIG. 2B shows a view of the mechanical subsystem as viewed from the front. Door 275 is vertically lowered to an open position thereby revealing portal opening 271. Through portal opening 271, collimator 280 and patient table 290 are visible within the interior. In this embodiment, door 275 is opened by lowering it into pit extension 254, using the door mechanism including door actuator 276 (e.g., a hydraulic jack) and using the space provided by this yet deeper portion of pit base 255. Such a configuration is advantageous as it reduces the clearance required around the portal opening for the door and associated movement mechanisms. It is appreciated that in various other embodiments, the door may be lowered from above or may be translated or rotated into position from any direction.

In this embodiment, the entire mechanical superstructure is linked together and supported by base ring 257, which substantially balances the massive loads of heavy shielding and other equipment on either side. Axial bracket 258 covers the axial shield and serves to cover essential electronics in a manner analogous to the oblique bracket on opposite side of the machine (not shown). Note that shell 265 is in the open position, where it has rotated about shell bearing 266 to underlie patient table base below patient table 290, leaving patient table 290 exposed from above. This position allows the patient table to roll outward to its full extent, enabling patients to be loaded and unloaded from the apparatus. Upon loading, patient table 290 rolls toward the collimator, shell 265 rotates about shell bearing 266 until the shell covers patient table 290, and door 275 on door actuator 276 raises into the closed and shielded position.

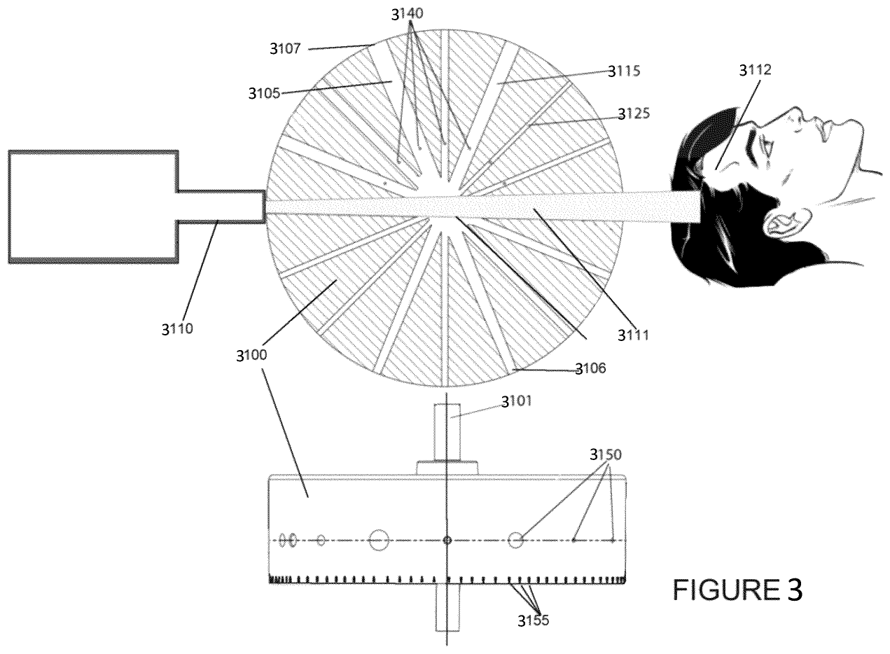

FIG. 3 shows a rotatable collimator that may be used with the multi-axis dynamic therapy system 100. In this embodiment, rotatable collimator is a revolving collimator wheel. FIG. 3 shows a cross-sectional view of a revolving collimator wheel 3100 having collimator channels 3140 passing therethrough. Collimator wheel 3100 has longitudinally extending channels or collimator channels 3105, 3115 and 3125 defined therein, for example machined through the body of collimator wheel 3100. The figure shows multiple other channels that are not labeled for the sake of clarity of the drawing. The collimator channels may be of various sizes, diameters, or shapes. In some embodiments, each collimator channel is of a different diameter. For example, as shown in FIG. 3, collimator channel 3105 is of larger bore than collimator channel 3115, which is of larger bore than collimator channel 3125. Each collimator channel extends from a radiation entrance aperture 3106 to an exit aperture 3107. In some embodiments, the size of the entrance aperture 3106 is smaller than that of the exit aperture 3107 to facilitate a sharp falloff of radiation dose at the margins of the irradiated area. The collimator wheel 3100 is rotated so that a selected collimator channel is aligned with a radiation source 3110 to allow passage of a particle radiation beam 3111 through the selected channel, thereby providing a desired therapy beam to a target 3112 in the patient. In a side profile of collimator wheel 3100 shown at the bottom of FIG. 3, the entrance and exit apertures 3150 are visible about the circumference collimator wheel 3100 turns on axis 3101. In this example, collimator wheel 3100, couples with a 50:1 reduction gearbox and electric motor. In some embodiments, channel 3105 and exit aperture 3107 are round. It is appreciated that in alternative embodiments, the channels may be of any size or shape, for example square. Collimator wheel 3100 may be formed of any suitable material, for example machined from a titanium alloy. While collimator wheel 3100 is shown as being oriented vertically relative to the surface on which the patient rests, it is appreciated that the collimator wheel 3100 may be configured in any orientation so long as the treatment beams passing through the collimator channels are directed to the target. Further, while the collimator wheel is shown as having eight collimator channels it is appreciated that such collimator wheels may include more or fewer collimator channels.

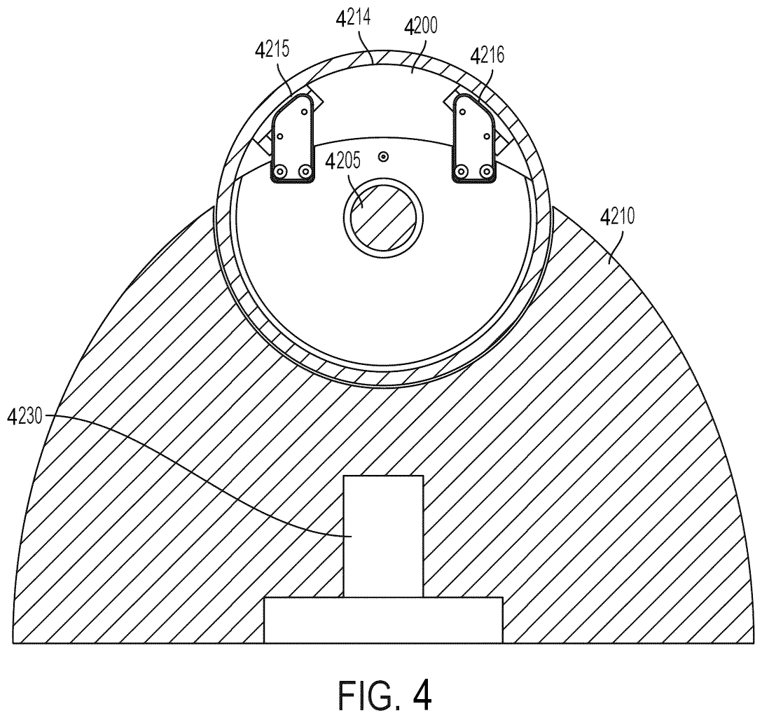

FIG. 4 shows a cross section of a revolving collimator wheel 4200 mounted upon a collimator shield 4210 and rotating between collimator positions on axis 4205. In this embodiment, the collimator shield 4210 substantially ensheaths the portion of the collimator wheel facing the radiation source so as to prevent radiation from entering non-aligned collimator channels, and of a thickness designed to otherwise provide shielding against leakage for the radiation source. The rotational position of the collimator wheel may be precisely controlled by a control system by using one or more sensors or encoders that monitor the position of the collimator wheel 4200, for example by detecting markers disposed on the periphery of the collimator wheel. In this embodiment, the position of the collimator wheel is precisely monitored by encoder reader heads 4215 and 4216, which track a thin tape-like encoder strip affixed to the internal rim 4214 of collimator wheel 4100, adjacent to the path of reader heads 4215 and 4216. The control system detects signals produced by precisely placed changes in the electromagnetic interaction between the encoders and the encoder strip. Using this combination of encoder strip affixed to internal rim 4214 and encoder reader heads, the control system senses when the wheel has been brought into the desired collimator position. Also shown in the cross section of collimator shield 4210 is a LINAC head 4230, which is the source of the radiation delivered to the entrance aperture of a selected collimator. In some embodiments, an ion chamber for measuring radiation intensity is included in the collimator shield between the LINAC head 4230 and collimator wheel 4200. Alternative positional encoder schemes may include mechanical stops such as gear teeth and divots, and/or optically sensed position markers.

In one embodiment, the collimator assembly and control system described above are incorporated into a treatment system. The control system includes a processor configured to facilitate controlled rotation of the collimator wheel to select positions corresponding to alignment of a selected collimator channel with the radiation source, the selected collimator channel corresponding to a desired treatment beam. In some embodiments, the treatment system includes a user interface that allows a treating physician to select one or more treatment beams associated with one or more collimator channels. In other embodiments, the control system automatically determine one or more collimator channels corresponding to a selected course of treatment.

FIG. 5 shows the apex of the conical shield, including the exit openings of the collimator channels and cameras for monitoring the patient. Collimator wheel 5300 is visible edge-on, including selected channel exit aperture 5305 and other apertures that are not identified and thus not aligned with the LINAC (underlying and not visible). Motor 5315 turns collimator wheel 5300 to a selected position in which the desired aperture is aligned with the LINAC. In some embodiments, the system includes one or more cameras to, for example cameras 5310, positioned to permit the patient to be monitored while undergoing radiation treatment. Encoder interpretation computer subsystem 5320 receives signals from encoder reader heads (see FIG. 4) to compute the precise rotational position of collimator wheel 5300, and hence the position of any of the selectable collimator channels. For example, in a feedback loop, motor control computer subsystem 5330 serves to activate motor 5315 until encoders indicate that the selected collimator is aligned with the LINAC. In one embodiment, the system is configured to change the collimator wheel position between multiple positions associated with selected collimator channels during therapy so as to differing sizes of treatment beams to the target.

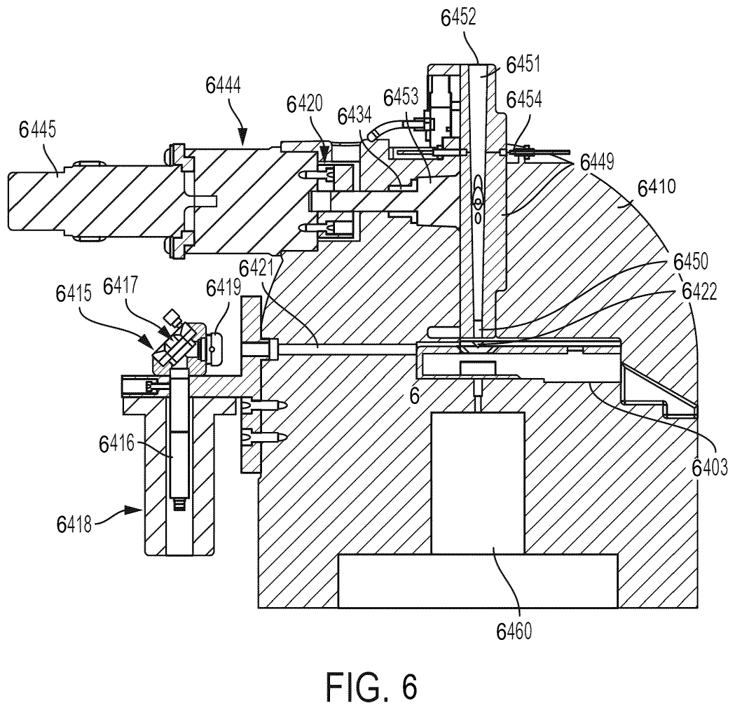

FIG. 6 shows a cross-sectional view of the collimator wheel in the context of the motor that drives between positions and a linear accelerator that passes radiation through an aligned collimator channel and associated features.

As shown, collimator wheel 6449 has selected and aligned channel 6451 with exit aperture 6452 and entrance aperture 6450. Collimator shield 6410 surrounds the collimator wheel 6449 along the portion facing the radiation source to allow passage of the particle beam through the inlet and exit orifices of channel 6451 while preventing radiation from entering non-aligned channels. In this embodiment, collimator wheel 6449 is selectively turned into the desired position via shaft 6453 with bushing 6434, shaft 6453 being connected with gearbox 6444 via coupling bracket and base 6420. Gearbox 6444 is coupled to and driven by motor 6445 and provides a reduction in revolutions at a pre-defined ratio, permitting very fine control of the degree to which the collimator wheel is turned and aligned with the radiation source, the LINAC head 6460 and distal margin of LINAC body 6461. The pre-defined ratio may be 10:1, 20:1, 30:1, 40:1, 50:1, 60:1, 70:1, 80:1, 90:1 or any ratio suitable for a given geometry of the collimator wheel and treatment system and desired resolution of adjustment. In this embodiment, energy exiting LINAC head 6460 enters sealed ion chamber 6403 which permits monitoring of dose, dose rate and field symmetry by virtue of the fact that radiation that enters the ion chamber will produce a measurable ionization current that is proportional to the x-ray beam intensity.

In this embodiment, mechanical alignment is optimized using optical beam techniques. This has the advantage of maximizing the transfer of radiation from the ion chamber 6403 into entrance aperture 6450. For this purpose, the system includes laser shield mount holding laser 6416, the beam from which is bent at a right angle by mirror 6415 and directed into diaphragm iris lens 6419 after which the laser light passes through shield bore 6421 defined in shield 6410 to reach the beam path right angle optical mirror 6422. Because the beam path right angle optical mirror 6422 is reflective to light but transparent to radiation, a properly aligned collimator may be detected by a laser beam being emitted from exit aperture 6452 of collimator wheel 6449, while maintaining functionality of the primary radiation delivery alignment, a function useful in initial validation and verification of each machine.

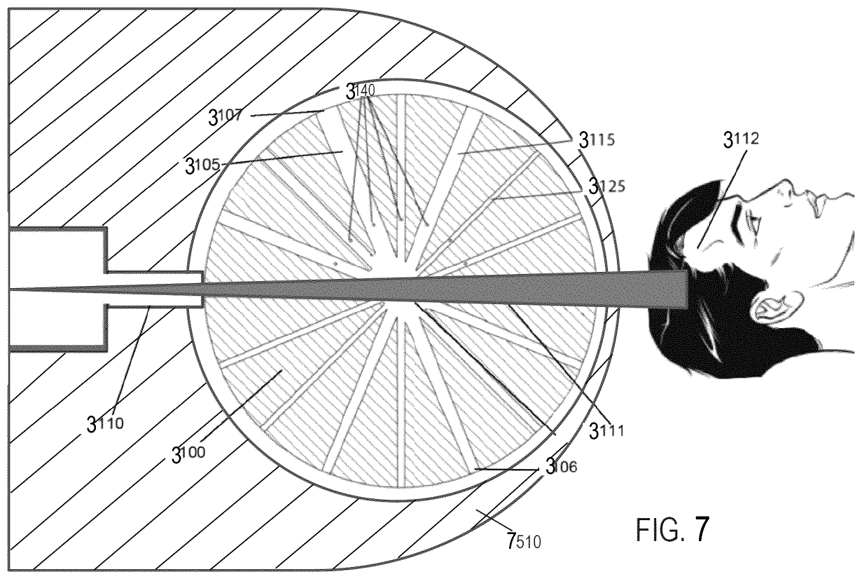

FIG. 7 shows another example system with the same or similar collimator wheel and associated components shown in FIG. 3 and a conical collimator shield 7510 that substantially surrounds the periphery of the collimator wheel so as to block and absorb radiation emitted from orifices of non-aligned collimator channels while allowing passage of a particle radiation beam 3111 from the radiation source 3110 through the selected, aligned collimator channel to provide a desired therapy beam to the target 3112. In this embodiment, the collimator shield 3510 covers any orifices of non-aligned channels, and its resulting greater thickness minimizes radiation leakage from linear accelerator (e.g., radiation source 3110). In some embodiments, the shield may cover less than all orifices of non-aligned channels. It is appreciated that in any of the embodiments herein, the shield may include multiple shield components to cover orifices of non-aligned channels and is not required to be a unitary component. Various configurations of the shield may be realized in accordance with the concepts described herein. While a collimator wheel has been shown and described here, it is appreciated that various other types of collimators may be used with the multi-axis dynamic therapy system as well, including multi-leaf collimators (MLC) or any suitable type of collimator.

In stereotactic radiosurgery, multiple advantages may be realized using dynamic delivery of radiation doses, e.g., the delivery of dose while the gantry is in motion. However, dynamic delivery introduces new challenges in treatment planning and treatment delivery. For example, the system must be able to accurately control radiation dose while the treatment gantry is moving around the patient. Embodiments of the present disclosure provide treatment plans including sequences of segments where each segment comes with a total dose to be delivered. At least some of the embodiments minimize treatment time by moving the gantry as fast as possible over the segments. In some embodiments, the gantry may slow down at or near certain angles to increase dose at the target.

Embodiments of the present disclosure describe dynamic beam delivery (DBD). DBD reduces treatment time by operating the LINAC radiation source while the gantries are moving the collimator. This results in the radiation beam moving with respect to the patient during treatment. The effect of the moving radiation beam may be approximated by a set of beamlets, e.g., small amounts of radiation delivered from a segment of the path along which the collimator moves. The dose is calculated by summing the radiation from each segment to produce the delivered dose. The segments may be made as small as necessary for accuracy. Each segment is computed as a Control Point (CP) that involves the effective gantry location for the segment along with the amount of radiation delivered at that point.

DBD paths may be based on the step-and-shoot paths used in non-dynamic deliveries. One method of creating step-and-shoot paths is to identify a large number of gantry/radiation source positions and allow an inverse-planning solver to determine which of these positions will be used for delivering radiation and the amount of radiation at each position. Many different inverse-planning solvers have been developed and may be used such as linear programming, but they generally define a set of beams that may create the specified dose distribution. These inverse planners select the positions where radiation is efficiently and effectively delivered to the target. Travelling Salesman solution methods may be used to create a path that efficiently visits the locations where radiation must be delivered. Dynamic Delivery may follow roughly the same path but deliver radiation while moving between beams. CPs would be defined along the path traversed by the radiation source and the amount of radiation (monitor units or MU) for each CP calculated with the same or different inverse planning solvers. This would define the MU to be delivered on the segment of the path running from one CP to the next. If the movement time in the leg is less than that required to deliver this dose, the movement time is increased to cover the excess dose delivery time. This excess time may be spent by slowing down the gantry move to allow the full dose to be delivered or by delivering some of the dose with the gantry stopped.

In some situations-particularly multi-Met-it may be advantageous to sacrifice conformity for treatment speed. For example, a single arc may be used per target with the linac running continuously to deliver the necessary dose in the minimum amount of time. For example, a circular arc using one or both axes of rotation may be used to deliver a near-spherical dose distribution to cover each target. In this situation, the time to treat each target would be dependent on the time required to deliver the prescribed dose to the target. Given limitations on the output of the LINAC, time may be reduced by choosing an arc that reduces the effective depth of the target.

Embodiments of the present disclosure balance the speed of delivery and the treatment plan quality. Speed of delivery is limited by the effective depth of the target while the plan quality is limited by the solid angle or diversity of angles from which radiation is delivered. According to methods described herein, a path is created that covers directions where the effective depth is low. CPs are placed on this path and a plan is created based at least in part on the CPs. The plan may be evaluated, and further iterations of path and CP weights may be created. If the treatment time is limited by gantry speed, the further iterations may focus on reducing the length of the path. If the treatment time is limited by dose rate, the further iterations may use a longer path to provide better plan quality.

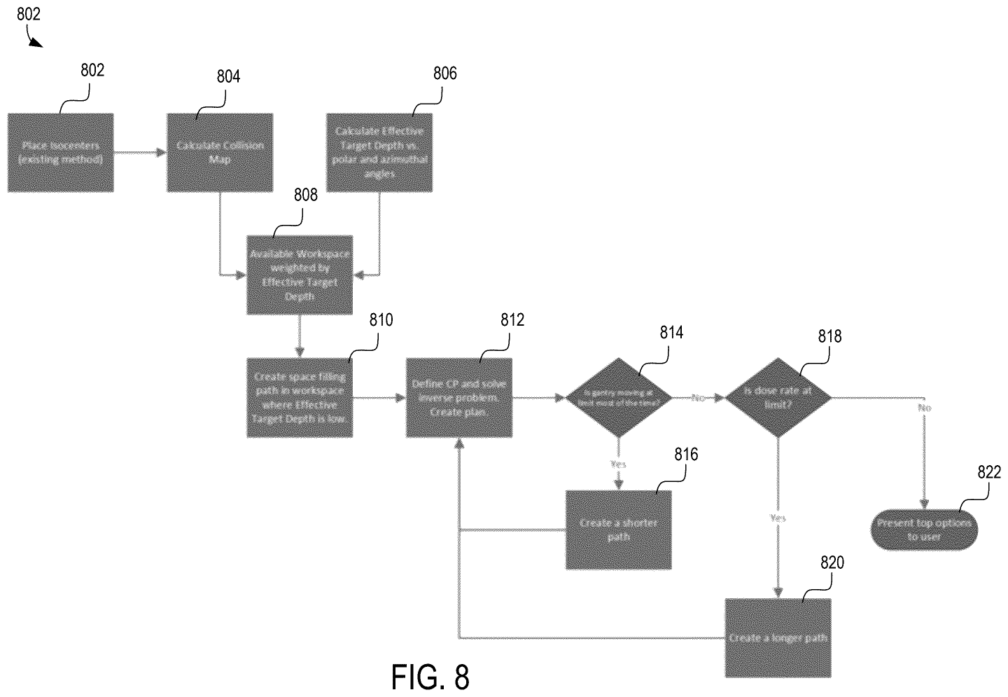

FIG. 8 is a flowchart of an iterative method for path selection. Method 800 is an iterative method for creating DBD paths. Method 800 describes operations for selecting the path to be travelled by the collimator. Method 800 may include more or fewer operations than those described herein. Various operations may be performed in alternative configurations than those described herein. Method 800 may include step 802 including placing isocenters according to existing methods such as. sphere packing to provide a variety of isocenter locations within the anatomical target. Step 804 includes calculating a collision map that limits the positions of the collimator relative to the patient in order to avoid collisions between the collimator and the patient. Step 806 includes calculating an effective target depth vs. polar and azimuthal angles. Target depth determines the efficiency of radiation delivery to the target. Greater depths result in more of the radiation being absorbed by overlying tissue and not reaching the target. The output of step 804 and step 806 are input to step 808 including determining the available workspace weighted by the effective target depth. Step 810 includes creating a space filling path in the workspace where the effective target depth is low to produce efficient radiation delivery. A space-filling path provides a wide variety of angles from which radiation may be delivered to the target. This variety allows dose gradients to be created by beams from different directions overlapping at the target to improve image quality. Step 812 includes defining one or more CPs along the path and solving the inverse problem by creating a plan by approximating the DBD from that path with a set of closely spaced beams at CPs along the treatment path. Plan quality delivery time may be optimized to some degree by adjusting the path length so that the radiation source is moving at its maximum speed and delivering radiation at its maximum rate for the majority of the treatment. Step 814 includes determining whether the gantry is moving at the limit most of the time. If yes, method 800 proceeds to step 816 including creating a shorter path. Method 800 iterates between step 812, step 814, and step 816 until the answer to step 814 is no. If no, method 800 proceeds to step 818 to determine whether the dose rate is at the limit. If yes, the method 800 proceeds to step 820 including creating a longer path. Method 800 iterates between step 812, step 814, and step 818 until the answer to step 818 is no. If no, method 800 proceeds to step 822 including presenting the top options for treatment paths to a user.

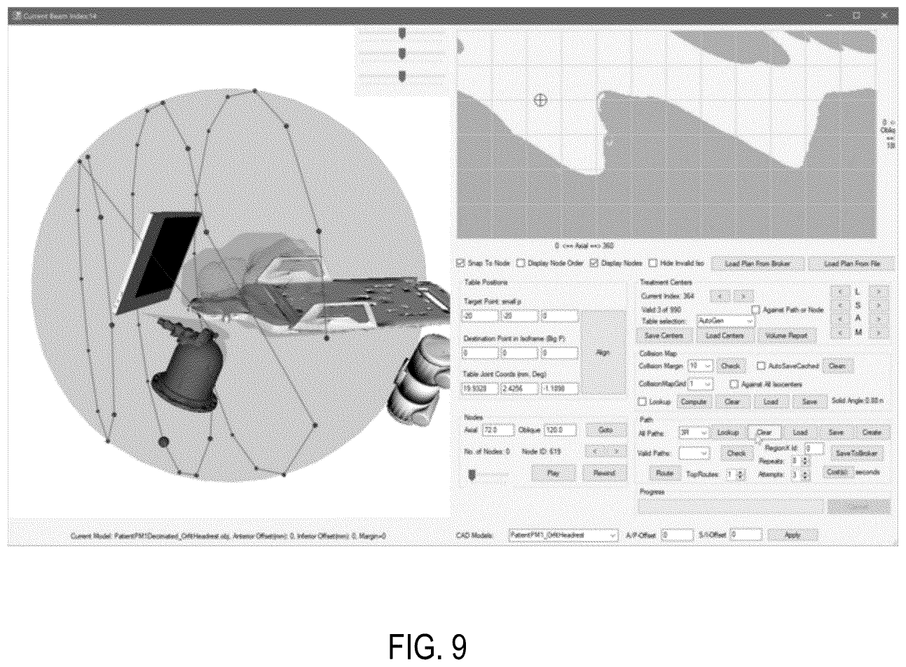

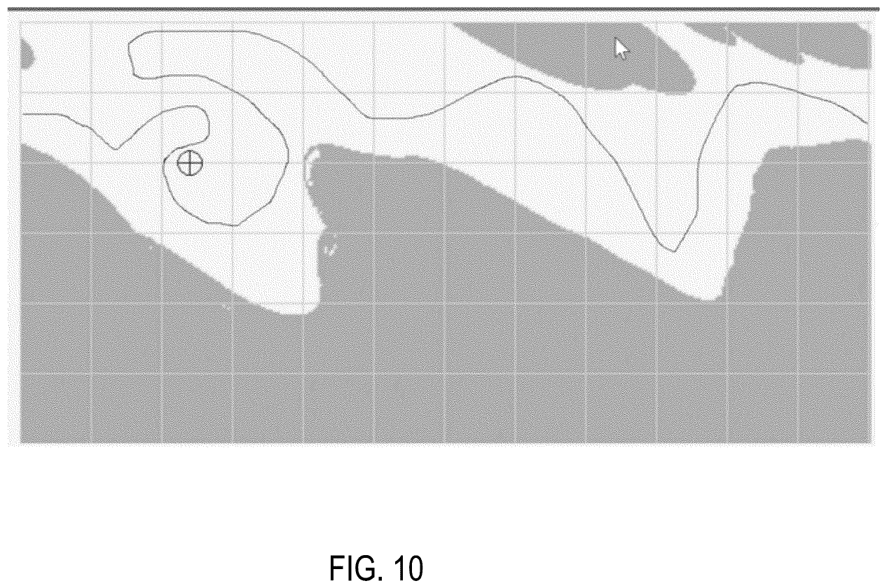

FIG. 9 is an exemplary complicated workspace having an isocenter in the neck. As shown in FIG. 9, an exemplary workspace includes an isocenter in the neck and the collision zone is spread across the available workspace into a more linear region. In these situations, it might be effective to combine a spiral near the point of minimum effective target depth (for example the gantry location shown) with a linear path to cover more solid angles. For example, FIG. 10 is an exemplary treatment path. FIG. 10 illustrates the linear region curving back and forth to cover more of the workspace in the linear region. The path used for DBD may be chosen based on the effective target depth for available gantry positions. Methods may be devised to place the path in locations where the effective target depth is small. The length of the path and how far from the minimum effective target depth reaches may be determined by iterating on the path selection and the resultant plan where the optimal plan will have the gantries moving near their maximum speed and with the LINAC delivering dose near its maximum rate for most of the path. Additional considerations for determining the path used for DBD include avoidance of critical structures and the shape of the resulting dose distribution near the target.

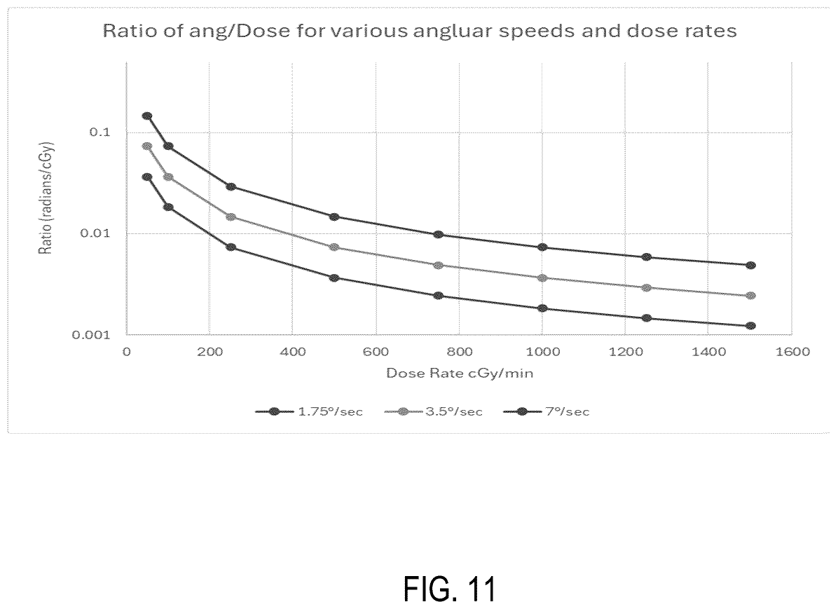

FIG. 11 is a graph of the ratio of angular motion to dose rate. FIG. 11 illustrates the ratio of angular motion to dose rate over speeds from 1.75°, 3.5° and 7° for dose rates from 200 cGy/min-1500 cGy/min. During the traversal of the path, the dose is delivered uniformly. To achieve this, the dose is adjusted to track the velocity of the gantry. Accordingly, a slow gantry velocity provides low dose delivery, and a high gantry velocity provides high dose delivery. The velocity inputs to the software are the respective velocities of the encoders for the rotation axes of the system.

In a gyroscopic system, complex calculations are performed to translate axis velocity inputs and axis positions into the velocity of the LINAC. This calculation may be performed in real-time at a high clock rate during treatment delivery, and the calculation algorithm may be optimized for speed. The speed of the gantry changes slowly in time due to the large moment of inertia. Various embodiments of the present disclosure provide a relationship between the dose required on the segment and the motion of the gantry. In some embodiments, the ratio is 7.33 rad/min/1500 cGy/min=4.887*10-3 rad/cGy.

A DBD treatment plan may include CPs that are defined by the radiation source location (gantry position) and the dose to be delivered in the segment connecting each CP to the next. Errors in radiation source location and dose rate are to be expected and feedback from position sensors (encoders) and dose monitors (ion chambers) will be compared to the planned CP and corrections generated. At each step in the delivery of a segment, a model may be based on ion chamber data of the instantaneous dose delivered. In some embodiments, there is a delay in the ion chamber of about 100 msec. At time T in the execution of the segment, the dose function may be integrated, and the total dose calculated to deliver up to 100 msec. The actual dose delivered may be compared to the integral of the prescription dose function as of time T-100 msec to ascertain the amount of over-delivery or under-delivery of dose at each point in time. In 100 ms, the number of pulses may range from 5 to 60 pulses. For example, at a nominal dose rate of 1500 cGy/min in 100 ms, the dose is 2.5 cGy. The gantry moving at a maximum angular rate would have moved 12.21 milliradians or 0.7 degrees.

In some embodiments, an incrementally updated quadratic model of the discrepancy between the predicted dose function and the observed dose function is constructed. The model may have a constant term, a proportional term, a quadratic term etc. The model may be used to adjust commands to the LINAC hardware as necessary to fit the delivered dose to the dose specified in the treatment plan. The model may use exponential damping, so that physical changes in the LINAC hardware that cause changes in the relationship between predicted dose and observed dose, and any changes may be incorporated into the model in a timely manner.

According to at least some embodiments, on-the-fly re-calculation of the treatment plan dose function for the segment based on the error detected in dose delivery at each point in time may be performed. For example, if at time T, 50% of the dose should have been delivered for the segment, but it is discovered that only 45% of the dose has been delivered, the remaining dose may be adjusted upward. In other embodiments, the gantry may be slowed down if the dose rate is already at the maximum dose rate. In this example, the remaining dose function may be increased upward by 10%, so that instead of giving the remaining 50% it would give 55%. The proportional increase of the entire remaining dose function spreads the adjustment uniformly over the remaining portion of the segment being delivered.

In some embodiments, the most recent 100 milliseconds may be considered a dose with an unknown error. Using the quadratic model, the observed dose delivery function may be extended from T-100 to time T. This provides an approximate model of the dose delivery function from time 0 (the start of the segment) to time T (the current instant in time). The integral of this function is used as the actual delivered dose in the above calculation.

In calculating the dose to be delivered at each time interval, the quadratic model may be used to relate the commanded dose to the delivered dose to accurately adjust the dose. In the process of incrementally updating the quadratic model, the model-output actual commanded dose may be used instead of the original prescription dose function from the treatment plan. The model captures the differences between the commands issued to the LINAC hardware and the measured dose that is delivered. In some embodiments, an incremental linear regression algorithm that permits the efficient update of matrices of fixed size as each new ordered pair of dose-delivered and dose-commanded values is received may be used.

According to some embodiment, incoming ordered pairs <commanded_dose, actual_dose> are given increased weights to bias the model parameter estimates toward more recent observations. Mathematically, this would be equivalent to adding multiple duplicate rows to the regression model being solved for the most recent incoming ordered pairs. Accordingly, the dose delivered to the patient is optimized for consistency and uniformity over each of the segments specified in the treatment plan.

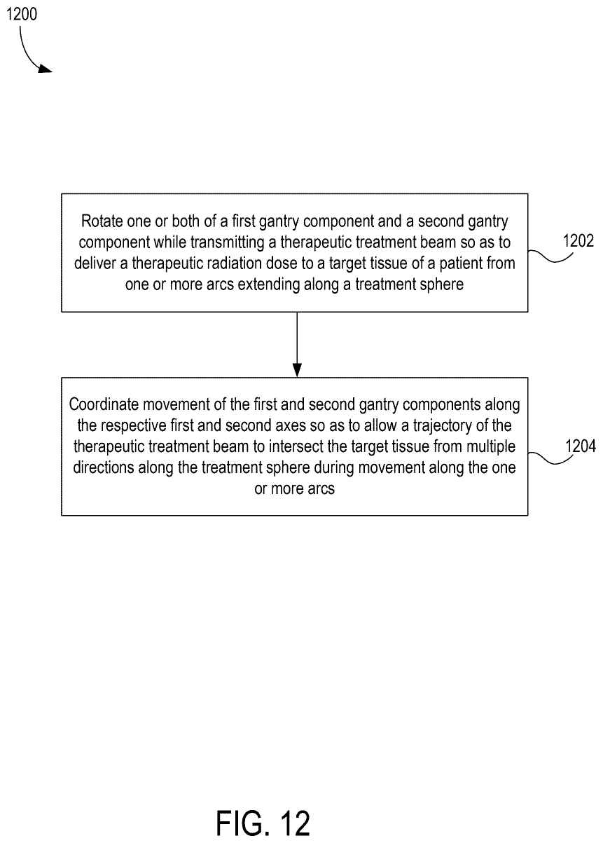

FIG. 12 is a flowchart of a method for treating a patient with a radiosurgical treatment system. Method 1200 implements various embodiments described in detail above such as the radiosurgical treatment system and collimator described with respect to FIGS. 1-5. Method 800 describes operations for operating the system to deliver VMAT treatment to target tissue in a patient. Method 1200 may include more or fewer operations than those described herein. Various operations may be performed in alternative configurations than those described herein.

Method 1200 may include step 1202. Step 1202 may include rotating one or both of a first gantry component and a second gantry component while transmitting a therapeutic treatment beam so as to deliver a therapeutic radiation dose to a target tissue of a patient from one or more arcs extending along a treatment sphere. In various embodiments, the first gantry component is rotatable about a first axis that extends along a patient supported within an interior treatment space and a second gantry component is interfaced with the first gantry component such that rotation of the first gantry component rotates both the first and second gantry components about the first axis. In some embodiments, the second gantry component is independently rotatable about a second axis that is transverse to the first axis and intersects the first axis at an isocenter. This allows for treatment angles along a majority of a treatment sphere which is far greater than treatment angles accessible by conventional systems that are coplanar or more limited in axes of rotational movement. According to various embodiments, systems and methods of the present disclosure may treat with non-coplanar arcs. The system and methods describe herein may arcs like in a C-arm systems but with additional degrees of freedom as described above such that more solid angle coverage in the form of non-coplanar arcs is provided.

Method 1200 may further include step 1204. Step 1204 may include coordinating movement of the first and second gantry components along the respective first and second axes so as to allow a trajectory of the therapeutic treatment beam to intersect the target tissue from multiple directions along the treatment sphere during movement along the one or more arcs. In some embodiments, the one or more arcs are non-coplanar from each other. This approach allows for more precision delivery of high doses to the target while minimizing irradiation of healthy tissues, particularly critical structures, including but not limited to the optic nerve. This approach also allows for more efficient treatment systems as the therapy may be delivered from more treatment angles without requiring repositioning of the patient.

Various embodiments of method 1200 may include portions of the treatment that are static or dynamic. Dynamic deliveries may decrease treatment time for the patient on the treatment table and spreading the dose over a much larger volume of healthy tissues to spare more negative sequalae to patients. In some embodiments, the dose is better delivered via a mix of fixed and dynamic fields.

Method 1200 may further include rotating a collimator wheel mounted within the at least one of the first and second gantry components and in line with the therapeutic treatment beam. The collimator wheel may rotate along a rotation axis that is perpendicular to an axis of the therapeutic treatment beam. The collimator wheel may direct the therapeutic treatment beam passing through the collimator wheel to the isocenter for treatment of a target positioned within the treatment sphere. In at least some embodiments, the first and second gantry components are may be rotated independently from the rotation of the collimator wheel.

The collimator wheel may include various embodiments. The collimator wheel may include a plurality of collimator channels. The plurality of collimator channels may be arranged substantially perpendicular to a rotation axis of the collimator wheel. Accordingly, the method may include rotating the collimator wheel to align with one or more collimator channels of the plurality of collimator channels with the therapeutic treatment beam. The one or more selected collimator channels may correspond to one or more desired therapy beams along the one more arcs of the treatment sphere.

In some embodiments, method 1200 includes rotating the collimator wheel to align with a different one or more collimator channels during treatment. The therapeutic treatment beam may be gated during rotation of the collimator wheel between channels. The different one or more collimator channels is an adjacent channel on the collimator wheel relative to the one or more collimator channels. For example, a nearest neighbor collimator channel may be selected during treatment and the therapeutic treatment beam may be gated during the transition from the first collimator channel to the second collimator channel.

According to various embodiments, method 1200 may include adjusting a speed of rotation for each of the first gantry component, the second gantry component, and the collimator wheel. For example, the speed of rotation at least one of the first gantry component, the second gantry component, and the collimator wheel is proportional to a dose of therapeutic beam delivery to the target tissue.

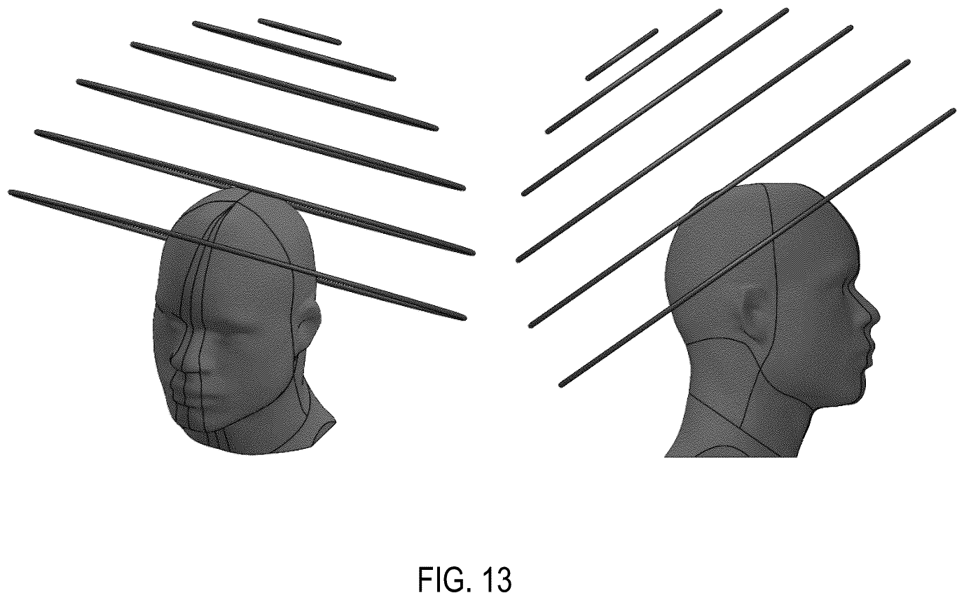

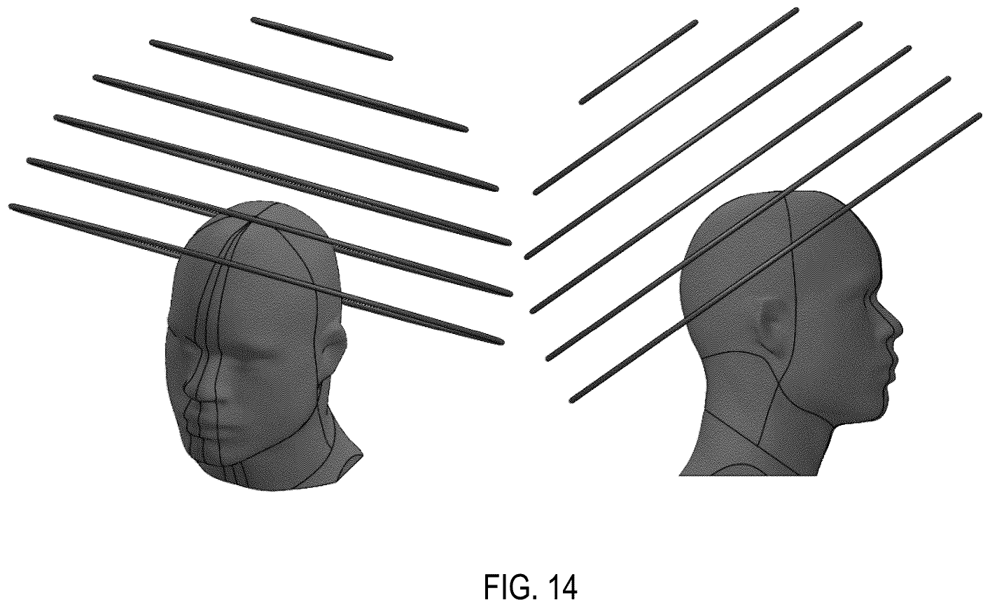

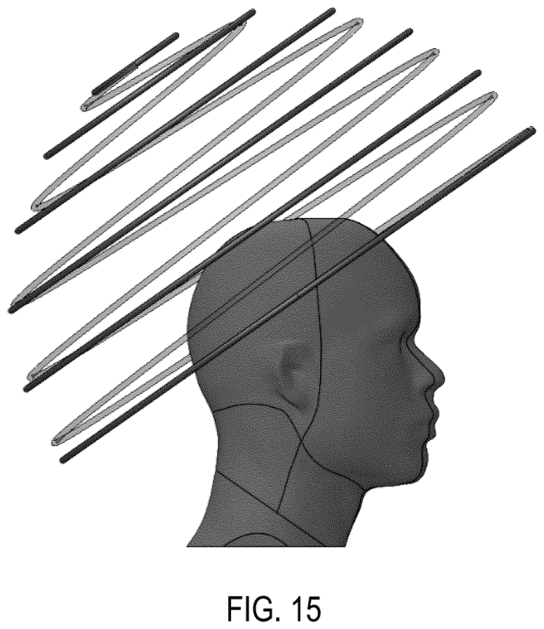

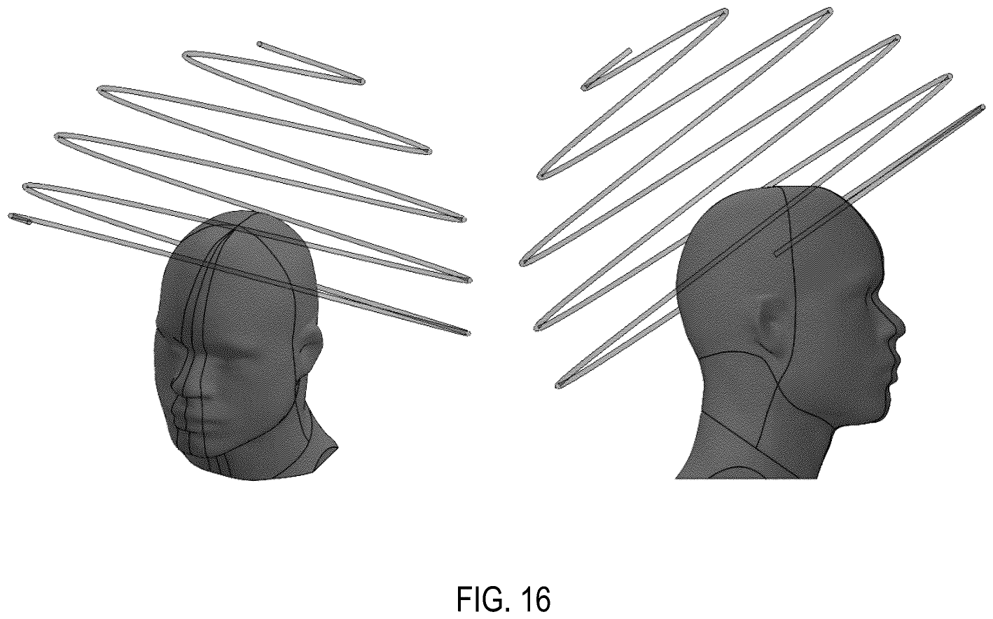

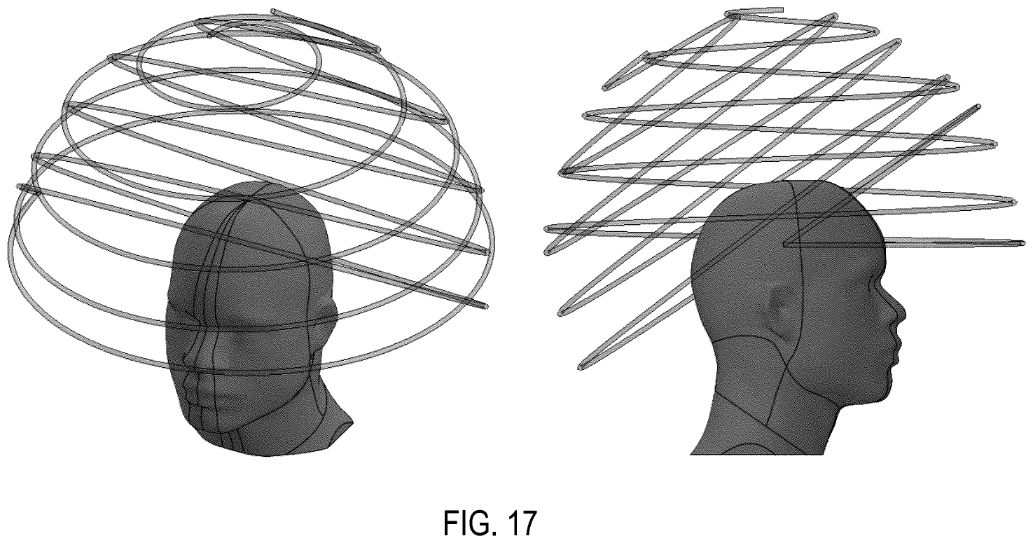

In various embodiments, a path of rotation for at least one of the first gantry component and the second gantry component includes a line, an arc, a circle, a great circle, a spherical spiral, a helical spiral, a spherical spiral, a rhumb line, etc., or any combination thereof. Exemplary and non-limiting treatment paths are illustrated in FIGS. 13-17. Another embodiment may include moving the patient table move during the delivery of the treatment beam. In this method, the tumor is moved by the table while both gantries are moving all while the treatment beam is delivering therapeutic radiation. During this delivery, any single or all axes may be moved with a high degree of coordination. The control system will monitor all motions and radiation delivery and will adjust dose or position as previously described to ensure the desired radiation delivery has been achieved.

According to some embodiments, the system further includes a patient table for supporting at least a portion of the patient having the target tissue within the interior treatment space. The patient table may be translated along at least three axes, thereby adding more degrees of freedom to the treatment system. Accordingly, the target tissue may include one or more distinct regions within a portion of the patient where the patient table is translated to treat different regions of the target tissue. The patient table provides more degrees of freedom and further enables the system to treat multiple regions of target tissue, for example, for metastatic tumors or the like. In some embodiments, the target tissue may include 3 or more tumor sites to be treated (e.g., 5 or more, 8 or more, 10 or more, 20 or more sites). In some embodiments, the higher throughput provided by the system allows for treatment of up to 20 sites in less than an hour. Furthermore, the patient table may provide a wobble to the isocenter for increasing the dose applied at certain angles at the tumor.

In yet further embodiments, the system includes a head rest or head support disposed on the patient table for maintaining an extension or flexion of a head of the patient. A head rest support for maintaining a patient's neck head extension (chin anterior) or flexion (chin posterior) may be used to increase the amount of solid angle for treatment of certain tumor sites. For example, by rotating the head to move the chin up, interference between the system (e.g., collimator) and the patient's chest may be reduced enabling radiation from inferior-anterior oblique directions. A head rest for maintaining a patient's head extension or flexion may be used to increase the amount of solid angle for treatment of certain tumor sites. The amount of flexion or extension may be patient specific and shall be determined by the neurosurgeon. For example, younger patients will generally have a much larger range of motion than elderly patients. Patients with prior injuries might have a limited range of motion. A health care provider may decide whether, and to what degree, to use such a head rest in combination with embodiments of the present disclosure. The amount of flexion or extension may be monitored by measuring the angular range of the head rest about a fixed point. According to some embodiments, an image tracking/guidance system may account for the new head and neck positions using a deformation single axis rotation correction of the current location when compared to the neutral head position which was used to acquire the CT or MRI imaging data set. The head rest may be used to vary the patient's head position over a few finite ranges of positions for example, the maximum extent, neutral, or the maximum flexion. The imaging system may account for these new positions and not interpret them as unintended patient motions. In some embodiments, the head rest is an adjustable, hinged head support attached to or otherwise disposed on the patient table that permits both extension and flexion of the neck of the patient in the sagittal plane, wherein the target tissue is within the head of the patient.

FIGS. 13-17 illustrate various exemplary treatment paths. Azimuthal arcs may not be the most efficient at delivering the dose because they do not minimize effective target depth for some treatment plans. A circular arc centered on the gantry position with the minimum effective depth may be more efficient (e.g., where the radiation is delivered from the side of the head with less effective depth (assuming that effective target depth varies slowly with gantry position)). A small radius delivers beams closer to the point with minimum effective depth and suffers less attenuation. However, the beams from the smaller radius will converge at the isocenter with a smaller angle, thereby resulting in lower gradients. For small radii, the length of the arc path will be short enough that the dose rate will necessitate a slow speed from the gantry. Increasing the radius of the path may increase the treatment time by the difference in the effective target depth between the two paths. At some radius, multiple circular arcs may be preferred to one large arc. This provides a diversity of angles by filling in the solid angle bounded by the outer circular arc.

In some embodiments, an irregular delivery path may be selected in order to best deliver radiation to the target area while sparing particular sensitive tissues. In this method, the Control Points will be designed and connected with arc segments including connecting splines and curves which do not exceed the physical performance criteria for the gantries and radiation dose to change direction or dose.

In some embodiments, an Archimedean spiral may be used to deliver dose from the position with minimum effective depth at the center of the spiral. The dimensions of the spiral would be constrained by the available workspace with deviations occurring to avoid collision zones and positions outside the range of motion of the gantry (e.g., polar angle less than 45 degrees or more than 135 degrees). For treatments with multiple isocenters, one isocenter would spiral into the gantry position with minimum target depth. The next isocenter would spiral out from this position. A transitional alignment may occur with the gantry at the middle of the spiral and the edge of the spiral on alternating isocenters.

In at least some embodiments, a Fermat's spiral is used. A Fermat's spiral advantageously starts and ends in approximately the same location. It may also be beneficial to avoid starting or stopping at the ideal gantry position for delivering dose. Spiral paths have an angular orientation which may be thought of as the direction at r=0. This may be chosen based on the gradient of the effective target depth at the point of minimum effective target depth. For example, in the Fermat's spiral shown above, it delivers along the x-axis near the middle. This spiral may be rotated so that this axis is aligned with the direction in which the effective target depth slowly increases.

According to various embodiments, a path may include a path of at least one of the patient table axes including a line, an arc, or an arbitrary spline and is proportional to a dose of therapeutic beam delivered to the target tissue.

According to some embodiments, during approaching and control point moving left to right on a control segment at point cᵢ.
1. LHS(cᵢ)=RHS (cᵢ) Continuity of the path moving from one function or spline to another
2. LHS′(cᵢ)=RHS′ (cᵢ) the first derivative is continuous, so the slopes of the tangent lines are the equal
3. LHS″(cᵢ)=RHS″ (cᵢ) the second derivative is continuous, so the concavity of the tangent lines is the equal.

This is for the spatial terms. This criterion provides smooth curves with no ill-defined paths.

If taking a derivative as function of time then,
1. LHS (cᵢ)=RHS (cᵢ) Continuity of the path moving from one function or spline to another
2. LHS·(cᵢ)=RHS·(cᵢ) the first derivative with respect to time is continuous, so the slopes of the tangent lines are the equal. This is an angular velocity.
3. LHS··(cᵢ)=RHS··(cᵢ) the second derivative with respect to time is continuous, so the slopes of the tangent lines are the equal. This is an angular acceleration.
4. LHS···(cᵢ)=RHS···(cᵢ) the third derivative with respect to time is continuous, so the slopes of the tangent lines are the equal. This is called the jerk which means the derivative of the acceleration.

Various embodiments of the present disclosure provide therapeutic treatment delivery with high precision and improved speed (e.g., reduced treatment time) compared to traditional delivery systems. For example, embodiments of the present disclosure provide treatment between 30% and 40% faster than conventional delivery with a higher throughput than previous point-and-shoot systems. Embodiments of the present disclosure aim to increase the RPMs of the gantry from 1 RPM to 2 RPM for further decreasing treatment times. Furthermore, embodiments of the present disclosure facilitate treatment of multiple sites, which is particularly advantageous for treatment of metastatic cancer. In some embodiments, 20 or more metastatic tumors may be treated in under an hour. Embodiments describe herein advantageously provide 7 degrees of freedom for treating tumors efficiently from all angles (e.g., the gantry components, the patient table, the collimator, the dose rate, etc.). All degrees of freedom are synchronized according to the present disclosure.

Embodiments of the present disclosure include adjusting the collimator during the motion of the gantry to use the linac acting as an MV imager to gain a limited view image of the patient. For example, a health care professional may have a limited field of view for locating bony landmarks or edges and matching the landmarks with various anatomical structures for guiding treatment and/or positioning a patient. In at least some embodiments, Hounsfield Units from clinical CT scan data may be used in the image reconstruction. When doing MV imaging with the linac, at least some embodiments do not deliver treatment energy during the imaging. Performing MV imaging with the linac may be performed concurrently and/or sequentially with kV imaging or other imaging techniques.

In some embodiments, a limited digital tomosynthesis (DT) reconstruction may be generated along a limited path of movement along an arc. DT is an imaging technique that may be used for head and neck imaging, thereby offering advantages over traditional radiography and potentially CT in certain situations. It provides cross-sectional images with lower radiation dose compared to CT, while still offering better visualization of structures compared to standard X-rays. Digital tomosynthesis captures multiple low-dose X-ray images from different angles. These images are then processed to reconstruct 2D or 3D images of a specific slice of tissue, like CT but with a lower radiation dose. DT delivers a lower radiation dose compared to CT scans, making it a potentially safer option for repeated imaging or for patients who need to avoid high radiation exposure.

Advantageously, DT offers better visualization of certain structures compared to traditional radiography, especially in areas with overlapping anatomy, like the paranasal sinuses. For example, DT may provide <1 mm accuracy depending on the detector and imaging system. In head and neck treatment setups, DT may achieve a positioning accuracy of less than 1 mm when bony structures are used as reference landmarks. See Junan Zhang, Q. Jackie Wu, Devon J. Godfrey, Toyosi Fatunase, Lawrence B. Marks, Fang-Fang Yin, “Comparing Digital Tomosynthesis to Cone-Beam CT for Position Verification in Patients Undergoing Partial Breast Irradiation, International Journal of Radiation Oncology*Biology*Physics, Volume 73, Issue 3, 2009, Pages 952-957, ISSN 0360-3016, https://doi.org/10.1016/j.ijrobp.2008.10.036., the disclosure of which is incorporated herein by reference in its entirety for all purposes.

According to various embodiments, MV imaging and techniques described herein may be used in various treatment planning and guidance applications. For example, DT may be used for on-board patient positioning in radiation therapy, thereby ensuring accurate targeting of tumors in the head and neck region. In another example, DT may be used in head and neck cancer treatment applications such as for treatment planning and daily patient positioning in radiation therapy for head and neck cancers, thereby improving accuracy and potentially reducing treatment margins.

According to at least some embodiments, treatments and treatment plans as described herein may include use of hybrid treatment modalities. Treatments and treatment plans may include any combination of step and shoot plans and continuous motion plans (e.g., full gyroscopic motion plans as described herein). In some embodiments, plans that use both step and shoot and continuous motion may be able to achieve the desired dose distributions in minimal time. Cases involving multi-metastatic tumor plans having larger tumors that are nearby each other may further benefit from the combination of different modalities.

Gantry motion with linac control and dose modulation may be used to generate spatially fractionated radiotherapy treatments. Spatially Fractionated Radiation Therapy (SFRT) is a specialized form of radiation therapy that delivers radiation in a non-uniform pattern, creating regions of high and low dose within the treatment area. This technique aims to maximize tumor control while minimizing damage to surrounding healthy tissues. It is particularly useful for treating large, radioresistant tumors and may potentially enhance the effectiveness of immunotherapy. Unlike conventional radiation therapy that delivers a uniform dose, SFRT creates a “peak-and-valley” pattern, with areas of high dose (peaks) and low dose (valleys) within the tumor volume. The high-dose regions target and destroy cancer cells, while the low-dose regions may allow healthy cells to recover and potentially stimulate an immune response. SFRT may be used to de-bulk larger tumors down to smaller more manageable volumes. By minimizing the overall dose to surrounding healthy tissues, SFRT may reduce side effects compared to conventional radiotherapy. Systems described herein having low leakage and sharp beam distributions may be further enhanced by these techniques. The low-dose regions may promote the migration of immune cells into the tumor, potentially enhancing the body's natural defenses against cancer.

Various techniques are used to achieve the spatially fractionated dose distribution. In some embodiments, GRID therapy uses a physical block with holes to create a pattern of radiation. In other embodiments, Lattice SFRT delivers radiation in a three-dimensional pattern of high-dose spheres, creating a more homogeneous low-dose distribution. Each of these techniques, or a combination of these techniques, may be used to create the grid pattern (2D) or a lattice pattern (3D) by moving a beam via gantry motion and controlling an ON/OFF pattern from the linac. This may be done is a step and shoot mode and/or a continuous mode. Additional details describing GRID therapy are described in “VMAT Grid Therapy: A Widely Applicable Planning Approach Grams,” Michael P. et al. Practical Radiation Oncology, Volume 11, Issue 3, e339-e347, the disclosure of which is incorporated herein by reference in its entirety for all purposes. Further details of Lattice SFRT are described in “Spatially fractionated stereotactic body radiation therapy (Lattice) for large tumors.” Adv Radiat Oncol. 2021 Jan. 8;6(3):100639. doi: 10.1016/j.adro.2020.100639.PMID: 34195486; PMCID: PMC8233471., the disclosure of which is incorporated herein by reference in its entirety for all purposes. According to various embodiments, GRID and/or SFRT patterns may be generated using any combination of step and shoot and/or continuous motion. For example, the beam may be gated between ON and OFF for building up the dose pattern.

Various relevant clinical applications may include the treatment of bulky and radioresistant tumors where SFRT is particularly useful for treating large, difficult-to-treat tumors that may be resistant to conventional radiation therapy. Other applications may include treating sarcomas which are often radio-resistant and may be challenging to control with standard treatments. Other clinical applications may include palliative care where SFRT may be used to relieve symptoms and improve quality of life in patients with advanced cancer. There is further potential for combination with immunotherapy. For example, the immune-stimulating effects of SFRT may make it a valuable tool in combination with immunotherapy.

Various embodiments of the present disclosure enable the collimator sizes to be changed during given segments to provide another degree of freedom for treatment optimization. This improves treatment plans where tumors are not often perfect geometric objects with clear boundaries. An optimal radiation field size may be chosen for the given treatment projection.

In some embodiments, TPS plans may be delivered on the equipment in an optimal way with minimal level of “jerk” smoothing for velocity and acceleration. Jerk is the time rate of change in acceleration. By looking at first, second and third directives in time of the motion the system moves between control points as quickly as possible while avoiding or reducing vibration issues for the large gantry which may cause table vibrations that the patient may feel or cause corrections on patient table location.

A system of one or more computers may perform particular operations or actions by virtue of having software, firmware, hardware, or a combination of them installed on the system that in operation causes or cause the system to perform the actions. One or more computer programs may perform particular operations or actions by virtue of including instructions that, when executed by data processing apparatus, cause the apparatus to perform the actions. One general embodiment includes a method of radiosurgical treatment. The method of radiosurgical treatment also includes rotating one or both of a first gantry component and a second gantry component while transmitting a therapeutic treatment beam so as to deliver a therapeutic radiation dose to a target tissue of a patient from one or more arcs extending along a treatment sphere, where the first gantry component is rotatable about a first axis that extends along a patient supported within an interior treatment space and a second gantry component is interfaced with the first gantry component such that rotation of the first gantry component rotates both the first and second gantry components about the first axis, where the second gantry component is independently rotatable about a second axis that is transverse to the first axis and intersects the first axis at an isocenter. The treatment also includes coordinating movement of the first and second gantry components along the respective first and second axes so as to allow a trajectory of the therapeutic treatment beam to intersect the target tissue from multiple directions along the treatment sphere during movement along the one or more arcs. Other embodiments of include corresponding computer systems, apparatus, and computer programs recorded on one or more computer storage devices, each may perform the actions of the methods.

Implementations may include one or more of the following features. The method where a path of rotation for at least one of the first gantry component and the second gantry component may include a line, an arc, a circle, a great circle, a spherical spiral, a helical spiral, or a rhumb line. The path of rotation is based on connecting beam positions chosen by inverse-planning optimization of at least one step and shoot plan to fill an available workspace. The path of rotation is an arc, where the arc traversed by the beam fills the available workspace near at least one position having a minimum target depth. The path of rotation is spherical spiral or a helical spiral that traverses spiral shapes to fill the available workspace. A length of the path of rotation is chosen to deliver the therapeutic radiation dose while moving the first gantry component and the second gantry component at maximum speed. The method may include rotating a collimator wheel mounted within the at least one of the first and second gantry components and in line with the therapeutic treatment beam, the collimator wheel may collimate the therapeutic treatment beam passing through the collimator wheel to the isocenter for treatment of a target positioned within the treatment sphere. The first and second gantry components may rotate independently from the rotation of the collimator wheel. The collimator wheel rotates along a rotation axis that is perpendicular to an axis of the therapeutic treatment beam. The collimator wheel may include a plurality of collimator channels, the plurality of collimator channels may include at least: a first collimator channel defined within the collimator wheel; and a second collimator channel defined within the collimator wheel, where the first and second collimator channels are arranged substantially perpendicular to a rotation axis of the collimator wheel. The method may include rotating the collimator wheel to align with one or more collimator channels of the plurality of collimator channels with the therapeutic treatment beam, the one or more selected collimator channels corresponding to one or more desired therapy beams along the one more arcs of the treatment sphere. The therapeutic treatment beam is gated during rotation of the collimator wheel between channels. The different one or more collimator channels is an adjacent channel on the collimator wheel relative to the one or more collimator channels. The method may include adjusting a speed of rotation for each of the first gantry component and the second gantry component. A dose of therapeutic beam delivery to the target tissue is proportional to the speed of rotation of at least one of the first gantry component and the second gantry component. The patient table may be translated along at least three axes. The target tissue may include one or more distinct regions within a portion of the patient, the method may include translating the patient table for treating a different region of the target tissue. The target tissue is within the head of the patient. The method may include performing lattice spatially fractionated radiation therapy (sfrt) and/or grid therapy. The method may include using the therapeutic treatment beam as a megavoltage (mv) imager. Implementations of the described techniques may include hardware, a method or process, or computer software on a computer-accessible medium.

Various embodiments include a radiosurgical treatment system. The radiosurgical treatment system also includes a radiation shield defining an interior treatment space, where the radiation shield may include: a first gantry component rotatable about a first axis that extends along a patient supported within the interior treatment space; and a second gantry component interfaced with the first gantry component such that rotation of the first gantry component rotates both the first and second gantry components about the first axis, where the second gantry component is independently rotatable about a second axis that is transverse to the first axis and intersects the first axis at an isocenter. The system also includes a radiation source disposed in at least one of the first and second gantry components to direct a continuous therapeutic beam to a target tissue within the interior treatment space. The system also includes a control unit operably and communicatively coupled with the radiation source, the first gantry component and the second gantry component, the control unit coordinates movement of the first and second gantry components along the respective first and second axes so as to allow a trajectory of the therapeutic beam emitted from the radiation source to intersect the target tissue from multiple directions along a treatment sphere. Other embodiments include corresponding computer systems, apparatus, and computer programs recorded on one or more computer storage devices, each may perform the actions of the methods.

Implementations may include one or more of the following features. The system may include a collimator wheel mounted within at least one of the first and second gantry components and in line with the radiation source, the collimator wheel may direct the therapeutic beam passing through the collimator wheel to the isocenter for treatment of a target positioned at the isocenter. The control unit may control movement of the first and second gantry components independently from the rotation of the collimator wheel. The control unit may control a speed of rotation for each of the first gantry component, the second gantry component, and the collimator wheel. A dose of therapeutic beam delivery to the target tissue is proportional to the speed of rotation of at least one of the first gantry component and the second gantry component. The control unit reduces the speed of rotation of the first gantry component and/or the second gantry component in order to deliver a dose to the control point. The collimator wheel is rotatable about a rotational axis thereof, the collimator wheel being circular in shape with a diameter, the collimator wheel having a plurality of collimator channels, the plurality of collimator channels including at least: a first collimator channel defined within the collimator wheel; and a second collimator channel defined within the collimator wheel, where the first and second collimator channels are arranged substantially perpendicular to the rotational axis of the collimator wheel. The control unit may rotate the collimator wheel to align with one or more collimator channels of the plurality of collimator channels with the radiation source, the one or more selected collimator channels corresponding to one or more desired therapy beams. The control unit may rotate the collimator wheel to align with a different one or more collimator channels during treatment, where the radiation source is gated during rotation of the collimator wheel during treatment. A path of rotation for at least one of the first gantry component and the second gantry component may include a line, an arc, a circle, a great circle, a spherical spiral, a helical spiral, or a rhumb line. The control unit is operably and communicatively coupled with the patient table for moving the patient table along at least three axes. The target tissue is within the head of the patient. The control unit may perform lattice spatially fractionated radiation therapy (sfrt) and/or grid therapy. The radiation source may be used as a megavoltage (mv) imager. The control unit adjusts a dose rate of the radiation source to synchronize the dose rate with the movements of the first gantry component and the second gantry component. The control unit adjusts a dose rate of the radiation source to correct for deviations in the planned movement of the first gantry component and the second gantry component across a control point. The control unit incrementally updates a quadratic model of a discrepancy between a predicted dose and an observed dose to adjust the dose rate and reduces the speed of rotation of the first gantry component and/or the second gantry component to minimize error. Implementations of the described techniques may include hardware, a method or process, or computer software on a computer-accessible medium.

While these components are shown in a particular arrangement in this example, it is appreciated that alternative configurations may be realized utilizing various other means of rotating the collimator wheel as would be understood by one of skill in the art. In addition, it is appreciated that certain elements may be omitted, such as the cameras, ion chamber and optical beam alignment features, while still retaining certain advantageous embodiments of the invention described above.

In the foregoing specification, the invention is described with reference to specific embodiments thereof, but those skilled in the art will recognize that the invention is not limited thereto. Various features and embodiments of the above-described invention may be used individually or jointly. Further, the invention may be utilized in any number of environments and applications beyond those described herein without departing from the broader spirit and scope of the specification. The specification and drawings are, accordingly, to be regarded as illustrative rather than restrictive. It will be recognized that the terms “comprising,” “including,” and “having,”as used herein, are specifically intended to be read as open-ended terms of art. Any patents, patent application or publications cited above are incorporated herein by reference in their entirety for all purposes.

1. A method of radiosurgical treatment, the method comprising:
rotating one or both of a first gantry component and a second gantry component while transmitting a therapeutic treatment beam so as to deliver a therapeutic radiation dose to a target tissue of a patient from one or more arcs extending along a treatment sphere, wherein the first gantry component is rotatable about a first axis that extends along a patient supported within an interior treatment space and a second gantry component is interfaced with the first gantry component such that rotation of the first gantry component rotates both the first and second gantry components about the first axis, wherein the second gantry component is independently rotatable about a second axis that is transverse to the first axis and intersects the first axis at an isocenter; and
coordinating movement of the first and second gantry components along the respective first and second axes so as to allow a trajectory of the therapeutic treatment beam to intersect the target tissue from multiple directions along the treatment sphere during movement along the one or more arcs.

2. The method of claim 1, wherein a path of rotation for at least one of the first gantry component and the second gantry component comprises a line, an arc, a circle, a great circle, a spherical spiral, a helical spiral, or a rhumb line.

3. The method of claim 2, wherein the path of rotation is based on connecting beam positions chosen by inverse-planning optimization of at least one step and shoot plan to fill an available workspace.

4. The method of claim 3, wherein the path of rotation is an arc, wherein the arc traversed by the beam fills the available workspace near at least one position having a minimum target depth.

5. The method of claim 2, wherein the path of rotation is spherical spiral or a helical spiral that traverses spiral shapes to fill the available workspace.

6. The method of claim 2, wherein a length of the path of rotation is chosen to deliver the therapeutic radiation dose while moving the first gantry component and the second gantry component at maximum speed.

7. The method of claim 1, further comprising rotating a collimator wheel mounted within the at least one of the first and second gantry components and in line with the therapeutic treatment beam, the collimator wheel being configured to collimate the therapeutic treatment beam passing through the collimator wheel to the isocenter for treatment of a target positioned within the treatment sphere.

8. The method of claim 7, wherein the first and second gantry components are configured to rotate independently from the rotation of the collimator wheel.

9. The method of claim 7, wherein the collimator wheel rotates along a rotation axis that is perpendicular to an axis of the therapeutic treatment beam.

10. The method of claim 1, further comprising adjusting a speed of rotation for each of the first gantry component and the second gantry component.

11. The method of claim 10, wherein a dose of therapeutic beam delivery to the target tissue is proportional to the speed of rotation of at least one of the first gantry component and the second gantry component.

12. The method of claim 7, wherein the collimator wheel comprises a plurality of collimator channels, the plurality of collimator channels comprising at least:
a first collimator channel defined within the collimator wheel; and
a second collimator channel defined within the collimator wheel,
wherein the first and second collimator channels are arranged substantially perpendicular to a rotation axis of the collimator wheel.

13. The method of claim 12, further comprising rotating the collimator wheel to align with one or more collimator channels of the plurality of collimator channels with the therapeutic treatment beam, the one or more selected collimator channels corresponding to one or more desired therapy beams along the one more arcs of the treatment sphere.

14. The method of claim 13, further comprising rotating the collimator wheel to align with a different one or more collimator channels during treatment, wherein the therapeutic treatment beam is gated during rotation of the collimator wheel between channels.

15 -20. (canceled)

21. A radiosurgical treatment system, the system comprising:
a radiation shield defining an interior treatment space, wherein the radiation shield comprises:
a first gantry component rotatable about a first axis that extends along a patient supported within the interior treatment space; and
a second gantry component interfaced with the first gantry component such that rotation of the first gantry component rotates both the first and second gantry components about the first axis, wherein the second gantry component is independently rotatable about a second axis that is transverse to the first axis and intersects the first axis at an isocenter; and
a radiation source disposed in at least one of the first and second gantry components and configured to direct a continuous therapeutic beam to a target tissue within the interior treatment space; and
a control unit operably and communicatively coupled with the radiation source, the first gantry component and the second gantry component, the control unit being configured to:
coordinate movement of the first and second gantry components along the respective first and second axes so as to allow a trajectory of the therapeutic beam emitted from the radiation source to intersect the target tissue from multiple directions along a treatment sphere.

22. The system of claim 21, further comprising a collimator wheel mounted within at least one of the first and second gantry components and in line with the radiation source, the collimator wheel being configured to direct the therapeutic beam passing through the collimator wheel to the isocenter for treatment of a target positioned at the isocenter.

23. The system of claim 22, wherein the control unit is configured to control movement of the first and second gantry components independently from the rotation of the collimator wheel.

24. The system of claim 22, wherein the control unit is configured to control a speed of rotation for each of the first gantry component, the second gantry component, and the collimator wheel.

25. The system of claim 21, wherein a dose of therapeutic beam delivery to the target tissue is proportional to the speed of rotation of at least one of the first gantry component and the second gantry component.

26. The system of claim 21, wherein a path of rotation for at least one of the first gantry component and the second gantry component comprises a line, an arc, a circle, a great circle, a spherical spiral, a helical spiral, or a rhumb line.

25.-37. (canceled)
